(19) United States
(12) Patent Application Publication
(10) Pub. No.: US 2020/0181595 A1
(43) Pub. Date: Jun. 11, 2020

(54) Bacillus Licheniformis Host Cell

(71) Applicant: Novozymes A/S, Bagsvaerd (DK)

(72) Inventors: Brian Koebmann, Herlev (DK); Michael Dolberg Rasmussen, Vallensbæk (DK)

(73) Assignee: Novozymes A/S, Bagsvaerd (DK)

(21) Appl. No.: 15/573,242

(22) PCT Filed: May 12, 2016
(86) PCT No.: PCT/EP2016/060713

§ 371 Date: Nov. 10, 2017

(30) Foreign Application Priority Data
May 12, 2015 (EP) 15167314.2

(51) Int. Cl. C12N 9/54; C12N 1/20

(52) U.S. Cl. CPC C12N 9/54; C12N 1/20

(57) ABSTRACT

The present invention relates to Bacillus licheniformis host cells producing heterologous polypeptide of interest, wherein at least one gene in the lan gene cluster inactivated and methods for producing the polypeptide of interest by cultivating said cells.

### REFERENCE TO SEQUENCE LISTING

This application contains a Sequence Listing in computer readable form. The computer readable form is incorporated herein by reference.

### FIELD OF THE INVENTION

The present invention relates to Bacillus licheniformis host cells producing a heterologous polypeptide of interest, wherein at least one gene in the lan gene cluster is inactivated and methods for producing the polypeptide of interest by cultivating said cells.

### BACKGROUND OF THE INVENTION

The complete genome sequences of several Bacillus species are in the public domain, see, e.g., Kunst et al., 1997, The complete genome sequence of the Gram-positive bacterium Bacillus subtilis, Nature 390, 249-256; Rey et al, 2004, Complete genome sequence of the industrial bacterium Bacillus licheniformis and comparisons with closely related Bacillus species, Genome Biol. 2004; 5(10):R77; and Veith et al, 2004, The complete genome sequence of Bacillus licheniformis DSM13, an organism with great industrial potential, J. Mol. Microbiol. Biotechnol. 7 (4), 204-211.

It has been reported that B. licheniformis contains a gene cluster in its chromosome that is believed to at least provide the potential for the bacterium to biosynthesise type II lantibiotics; (FIG. 1): structural genes are: lanA1, lanA2; lantibiotic modification genes are: lanM1, lanM2, lanB, lanC, lanP; regulation genes are: lanR, lank; transport genes are: lanT, lanP; and immunity genes are: lanE, lanF, lanG (Dischinger, J., Josten, M., Skekat, C., Sahl, H.-G., and Bierbaum, G. 2009. Production of the Novel Two-Peptide Lantibiotic Lichenicidin by Bacillus licheniformis DSM 13. PLos ONE, 4(8), e6788; Caetano, T., Krawczyk, J. M., Mösker, E., Süssmuth, R. D., and Mendo, S. 2011. Heterologous Expression, Biosynthesis, and Mutagenesis of Type II Lantibiotics from Bacillus licheniformis in Escherichia coli. Chemistry & Biology 18, 90-100).

One of the preferred workhorses in the recombinant production of polypeptides, especially of enzymes, is the prokaryotic bacterium Bacillus licheniformis. The industrial production of polypeptides is a competitive business, where even small incremental improvements in yield are highly desirable and where intense research activities are directed towards achieving this goal.

### SUMMARY OF THE INVENTION

In the examples provided herein it was demonstrated that inactivation of a gene in the putative lantibiotic biosynthesis gene cluster or inactivation of the entire lan cluster in a B. licheniformis host cell surprisingly resulted in a significant increase in the yield of a heterologous polypeptide enzyme of interest produced by said cell.

Accordingly, in a first aspect the invention provides a Bacillus licheniformis host cell producing a heterologous polypeptide of interest, wherein at least one gene in the lan gene cluster is inactivated.

In a second aspect, the invention provides a method for producing a polypeptide of interest, said method comprising a) cultivating a Bacillus licheniformis host cell as defined in any of the previous claims in a medium and under conditions conducive for the production of said polypeptide; and optionally b) recovering said polypeptide.

### BRIEF DESCRIPTION OF DRAWINGS

FIG. 1 shows the lan gene cluster in B. licheniformis. Structural genes are: lanA1, lanA2, lantibioic modification: lanM1, lanM2, lanB, lanC, lanP, regulation: lanR, lanK, transport: lanT, lanP, immunity: lanE, lanF, lanG.

FIG. 2 shows a schematic overview of the temperature sensitive plasmid vector pPP3932 for deletion of genes in B. licheniformis.

FIG. 3 shows the “Lig-PCR” of flanking regions upstream and downstream flanks to lanA1 in order to enable deletion of lanA1 in B. licheniformis.

FIG. 4 shows a schematic overview of the temperature sensitive plasmid pBKQ1697 for deletion of lanA1 in B. licheniformis.

FIG. 5 shows a schematic overview of the plasmid pBKQ1699 of Example 3.

FIG. 6 shows the “Lig-PCR” of flanking regions upstream and downstream flanks to the lan gene cluster in order to enable deletion of the entire lan gene cluster in B. licheniformis.

FIG. 7 shows a schematic overview of the temperature sensitive plasmid pBKQ1751 for deletion of the entire lan gene cluster in B. licheniformis. A region of res-cat-res is inserted in between the lan cluster flanks.

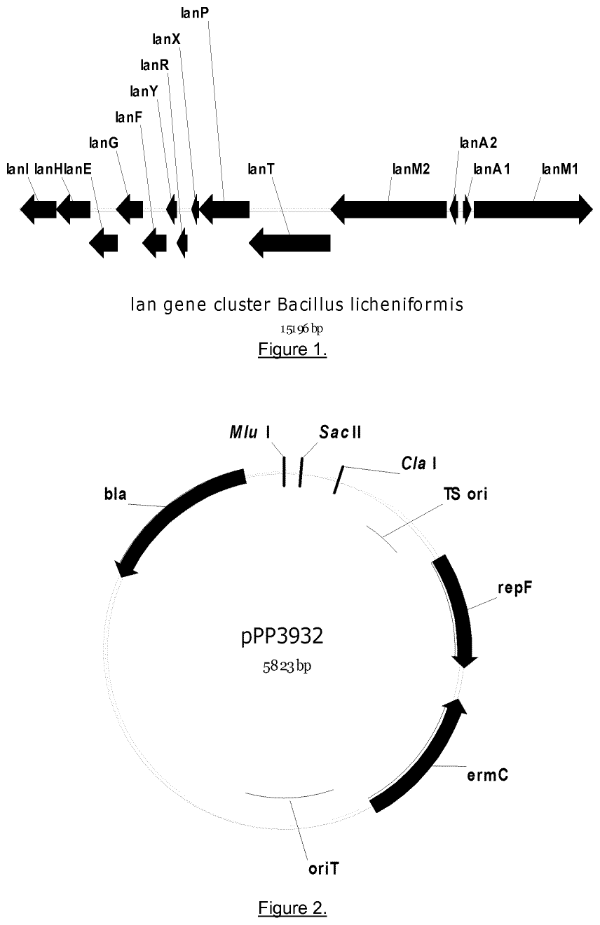

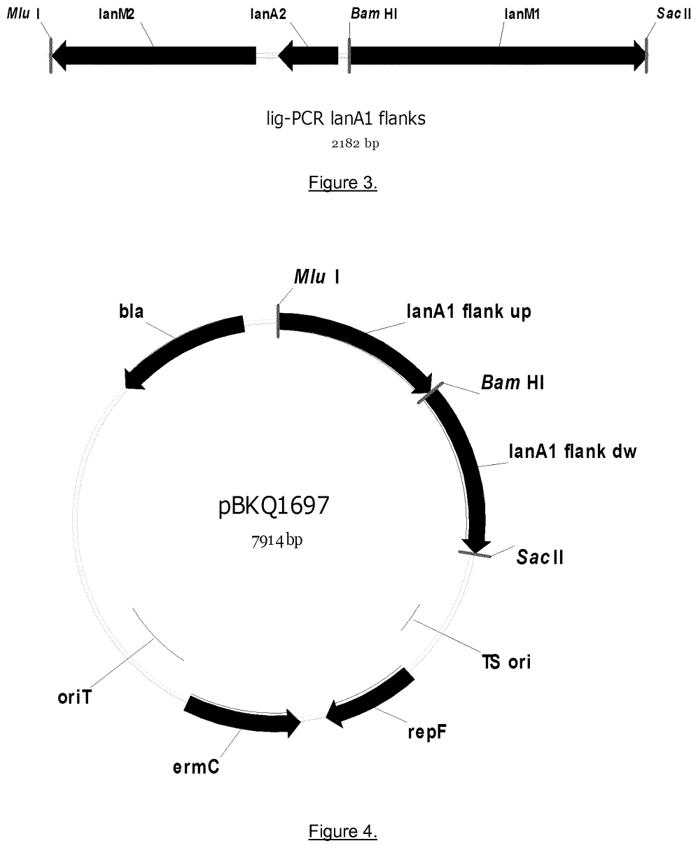

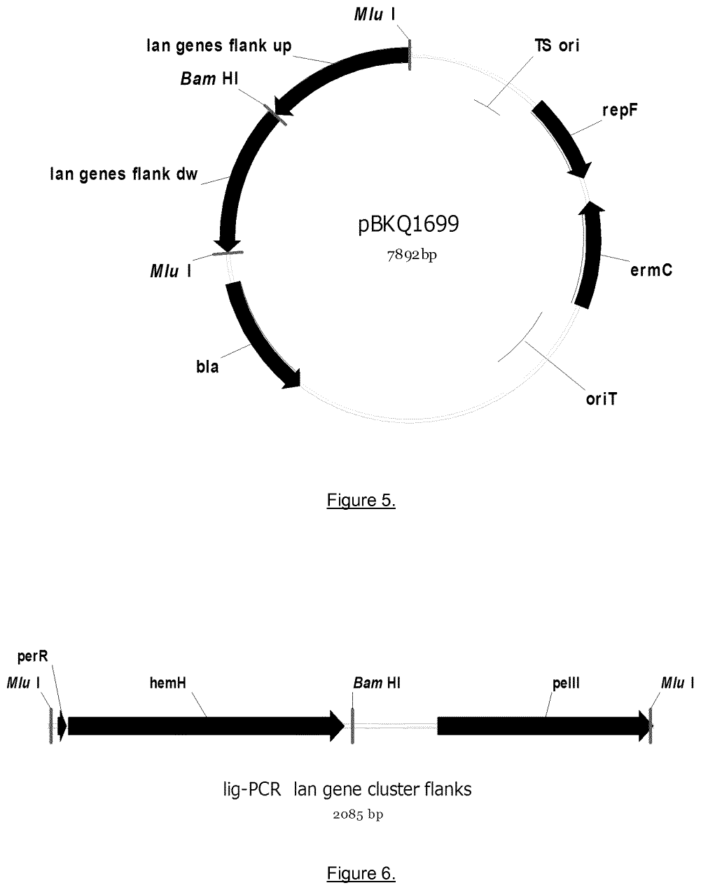

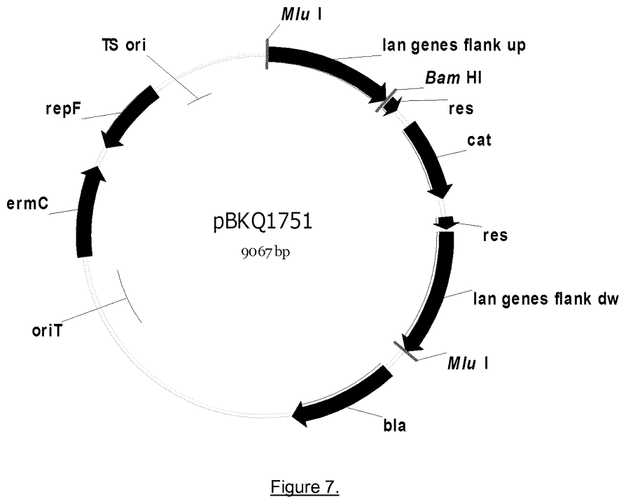

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

### DETAILED DESCRIPTION OF THE INVENTION

### Host Cells

The present invention relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector. The term “host cell” encompasses any progeny of a parent cell that is not identical to the parent cell due to mutations that occur during replication.

In a first aspect, the invention relates to a Bacillus licheniformis host cell producing a heterologous polypeptide of interest, wherein at least one gene in the lan gene cluster is inactivated.

In a preferred embodiment, the polypeptide of interest is expressed with or without a secretion signal peptide; more preferably the polypeptide of interest is secreted, non-secreted or intracellular. Expression in Bacillus of natively non-secreted polypeptides and natively secreted enzymes without a secretion signal peptide is disclosed, for example, in WO 2014/206829 or WO 2014/202793.

In a preferred embodiment, the polypeptide of interest is an enzyme; preferably, the enzyme is an oxidoreductase, transferase, hydrolase, lyase, isomerase, or ligase; preferably the enzyme is an aminopeptidase, amylase, asparaginase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, hyaluronic acid synthase, invertase, laccase, lipase, mannosidase, mutanase, oxidase, a pectinolytic enzyme, peroxidase, phytase, polyphenoloxidase, protease, ribonuclease, transglutaminase, or xylanase.

Stable expression of heterologous polypeptides in B. licheniformis host cells may be achieved through the integration of one or more copies of an expression construct in the chromosome of the host cell, for example, by transiently expressed phage-integrase mediated site-specific simultaneous integration in several loci as disclosed in WO 2006/042548.

Accordingly, in a preferred embodiment of the invention, the heterologous polypeptide of interest is encoded by an exogenous polynucleotide integrated into the chromosome of the host cell in at least one copy; preferably in at least two copies; more preferably in at least three copies; still more preferably in at least four copies; yet more preferably in at least five copies and most preferably in at least six copies.

There are many well-known ways to inactivate a gene, for example by mutating the gene through the introduction of a non-sense mutation or a frameshift mutation, or by partial or full deletion of the open reading frame, or by manipulation of one or more control sequence.

Accordingly, in a preferred embodiment of the invention, the at least one gene in the lan gene cluster is inactivated by a non-sense mutation in said at least one gene, a partial deletion of said at least one gene or open reading frame or a full deletion of said at least one gene or open reading frame.

It is well-known that Bacillus licheniformis species are very similar, so it is expected that other strains of that species will probably also have the lan gene cluster in their chromosome and it is expected that inactivation of one or more lan gene will have yield benefits as was demonstrated in the Bacillus licheniformis species employed in the examples herein. Even though the different Bacillus licheniformis species are similar, the DNA sequences of the lan genes may differ to some extent due to genetic variation or silent mutations.

Accordingly, in a preferred embodiment of the invention, the at least one gene in the lan gene cluster is selected from the group consisting of a lanI gene having a nucleotide sequence at least 70% identical to the lanI shown in SEQ ID NO:1, a lanH gene having a nucleotide sequence at least 70% identical to the lanH shown in SEQ ID NO:2, a lanE gene having a nucleotide sequence at least 70% identical to the lanE shown in SEQ ID NO:3, a lanG gene having a nucleotide sequence at least 70% identical to the lanG shown in SEQ ID NO:4, a lanF gene having a nucleotide sequence at least 70% identical to the lanF shown in SEQ ID NO:5, a lanY gene having a nucleotide sequence at least 70% identical to the lanY shown in SEQ ID NO:6, a lanR gene having a nucleotide sequence at least 70% identical to the lanR shown in SEQ ID NO:7, a lanX gene having a nucleotide sequence at least 70% identical to the lanX shown in SEQ ID NO:8, a lanP gene having a nucleotide sequence at least 70% identical to the lanP shown in SEQ ID NO:9, a lanT gene having a nucleotide sequence at least 70% identical to the lanT shown in SEQ ID NO:10, a lanM2 gene having a nucleotide sequence at least 70% identical to the lanM2 shown in SEQ ID NO:11, a lanA2 gene having a nucleotide sequence at least 70% identical to the lanA2 shown in SEQ ID NO:12, a lanA1 gene having a nucleotide sequence at least 70% identical to the lanA1 shown in SEQ ID NO:13 and a lanM1 gene having a nucleotide sequence at least 70% identical to the lanM1 shown in SEQ ID NO:14.

It is preferred that the at least one gene in the lan gene cluster is selected from the group consisting of a lanI gene having the nucleotide sequence shown in SEQ ID NO:1, a lanH gene having the nucleotide sequence shown in SEQ ID NO:2, a lanE gene having the nucleotide sequence shown in SEQ ID NO:3, a lanG gene having the nucleotide sequence shown in SEQ ID NO:4, a lanF gene having the nucleotide sequence shown in SEQ ID NO:5, a lanY gene having the nucleotide sequence shown in SEQ ID NO:6, a lanR gene having the nucleotide sequence shown in SEQ ID NO:7, a lanX gene having the nucleotide sequence shown in SEQ ID NO:8, a lanP gene having the nucleotide sequence shown in SEQ ID NO:9, a lanT gene having the nucleotide sequence shown in SEQ ID NO:10, a lanM2 gene having the nucleotide sequence shown in SEQ ID NO:11, a lanA2 gene having the nucleotide sequence shown in SEQ ID NO:12, a lanA1 gene having the nucleotide sequence shown in SEQ ID NO:13 and a lanM1 gene having the nucleotide sequence shown in SEQ ID NO:14.

In a preferred embodiment of the invention, two or more genes in the lan gene cluster are inactivated; preferably three or more genes in the lan gene cluster are inactivated; even more preferably four, five, six, seven, eight, nine, ten, eleven, twelve or thirteen or more genes in the lan gene cluster are inactivated.

Preferably, the genes in the lan gene cluster are inactivated by a non-sense mutation, a partial deletion or a full deletion of said genes, or by a combination thereof. It is preferred that the entire lan gene cluster is deleted.

### Methods of Production

The present invention relates to methods of producing a polypeptide in a host cell of the first aspect. The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

In a second aspect, the invention relates to a method for producing a polypeptide of interest, said method comprising:
a) cultivating a Bacillus licheniformis host cell as defined in the first aspect in a medium and under conditions conducive for the production of said polypeptide; and optionally
b) recovering said polypeptide.

### Sources of Polypeptides

The heterologous polypeptide of interest to be produced according to the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. For example, the polypeptide may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces polypeptide, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptide.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianurn, Trichoderma koningii, Trichoderma longibrachiatum, Trichoderma reesei, or Trichoderma viride polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to the expression of heterologous polynucleotides encoding the heterologous polypeptide of interest.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed, e.g., by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid expression constructs comprising a polynucleotide of the present invention operably linked to one or more control sequences that direct the expression of the coding sequence in a suitable host cell under conditions compatible with the control sequences to produce the heterologous polypeptide according to the invention.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausfi alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification.

### Expression Vectors

The present invention also relates to recombinant expression vectors comprising a polynucleotide encoding the heterologous polypeptide of interest according to the invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### EXAMPLES

### Materials and Methods

### Media

Bacillus strains were grown on LB agar (10 g/l Tryptone, 5 g/l yeast extract, 5 g/l NaCl, 15 g/l agar) plates or in LB liquid medium (10 g/l Tryptone, 5 g/l yeast extract, 5 g/l NaCl). To select for erythromycin resistance, agar media were supplemented with 2 to 5 μg/ml erythromycin and liquid media were supplemented with 5 μg/ml erythromycin. To select for chloramphenicol resistance, liquid and agar media were supplemented with 6 μg/ml chloramphenicol.

E. coli strains were grown on LB agar (10 g/l Tryptone, 5 g/l yeast extract, 5 g/l NaCl, 15 g/l agar) plates or in LB liquid medium (10 g/l Tryptone, 5 g/l yeast extract, 5 g/l NaCl). To select for erythromycin resistance, agar media were supplemented with 200 μg/ml erythromycin and liquid media were supplemented with 200 μg/ml erythromycin. To select for chloramphenicol resistance, liquid and agar media were supplemented with 6 μg/ml chloramphenicol.

To screen for protease phenotypes agar plates were supplemented with 1% skim milk to allow halos to form around the colonies that produces protease.

To screen for amylase phenotypes agar plates were supplemented with 1% starch to allow halos to form around the colonies that produces amylase.

Spizizen I medium consists of 1× Spizizen salts (6 g/l KH₂PO₄, 14 g/l K₂HPO₄, 2 g/l (NH₄)₂SO₄, 1 g/l sodium citrate, 0.2 g/l MgSO₄, pH 7.0), 0.5% glucose, 0.1% yeast extract, and 0.02% casein hydrolysate.

Spizizen II medium consists of Spizizen I medium supplemented with 0.5 mM CaCl₂, and 2.5 mM MgCl₂.

### Strains

E. coli TG1. Commercial strain used for cloning purposes (Stratagene).
B. subtilis PP3724. This strain is donor strain for conjugation of B. licheniformis as described in several patents (U.S. Pat. Nos. 5,695,976A, 5,733,753A, 5,843,720A, 5,882,888A, WO2006042548A1).
B. licheniformis SJ1904: This strain is a B. licheniformis strain described in WO 08066931 A2. The gene encoding the alkaline protease (aprL) is inactivated.
B. subtilis BKQ1707: This strain is PP3724 with pBKQ1697 for deletion of lanA1.
B. subtilis BKQ1754: This strain is PP3724 with pBKQ1751 for deletion of lan gene cluster.
B. licheniformis SJ12713: This strain is an alkaline protease AprH producing strain.
B. licheniformis BKQ1944: This strain corresponds to SJ12713 with deleted lanA1.
B. licheniformis BKQ1946: This strain corresponds to SJ12713 with deleted lan gene cluster.

### Primers

TABLE 1
Primer and sequence overview

| Primer | SEQ | |
|---|---|---|
| No./Seq. | ID NO | Nucleotide Sequence 5′→ |
| pr535 | 15 | GTGCTACGCGTGGGAATCTCCCAAATCCC |
| pr536 | 16 | GGTGAGGATCCGGAAAATTTCGATAGTTTGCCC |
| pr537 | 17 | CTTAAGGATCCCGCGTTGGCATATTGAT |
| pr538 | 18 | CGACACCGCGGAGGCGATAATGTTTTCG |
| pr539 | 19 | CGGAAACCGCTTTAGGGTTG |
| pr540 | 20 | GAGCCTGTGCAGCTGCAAG |
| pr541 | 21 | CATACTTTCTCCTCCTCTTTG |
| pr542 | 22 | CAAGATAGCGCATTTCGGG |
| pr601 | 23 | GCAGCTCCCTGTAATGTTCG |
| pr602 | 24 | CAGTAGACCGTACGGATCTG |
| pr547 | 25 | GTGCTACGCGTACAACATGCCAAGAACAGC |
| pr548 | 26 | GGTGAGGATCCATTGCAGCAAAAAGCGGAG |
| pr549 | 27 | CTTAAGGATCCAATCAAAATCTATGGATTTTCATC |
| pr550 | 28 | GCACAACGCGTAAATATGGCCTTCTCCGAA |
| pr551 | 29 | GGATCGCATCGATTGACGAG |
| pr552 | 30 | GCTGCCGATTTCTTCAGACC |
| pr553 | 31 | CAGACGGTAACCGTAACAAC |
| pr554 | 32 | GCCGCAATCAGCTGATCTCC |
| pr555 | 33 | CGAACTTTAAAGTGAACTCGCA |
| pr556 | 34 | CTCGAATTAATTCCGCTGTCG |

### Plasmids

pSJ3372: pUC derived plasmid with chloramphenicol marker from pC194 (U.S. Pat. No. 5,882,888)
pC194: Plasmid isolated from Staphylococcus aureus (Horinouchi and Weisblum, 1982, Nucleotide Sequence and Functional Map of pE194, a Plasmid That Specifies Inducible Resistance to Macrolide, Lincosamide, and Streptogramin Type B Antibiotics, J Bacteriol 150(2):804-814).
pPP3932 (SEQ ID NO:35): Temperature sensitive plasmid to be used for chromosomal replacement, mutation or deletion of B. licheniformis.
pBKQ1697 (SEQ ID NO:36): Plasmid pPP3932 with insertion of flanking regions of lanA1 from B. licheniformis SJ1904 in MluI and SacI site. The plasmid can be used for deletion of lanA1 in B. lichenformis SJ1904 derivatives.
pBKQ1699 (SEQ ID NO:37): Plasmid pPP3932 with insertion of flanking regions of lan gene cluster from B. licheniformis SJ1904 in MluI site.
pBKQ1751 (SEQ ID NO:38): Plasmid pBKQ1699 with insertion of res-cat-res region from pSJ3372 in between flanking regions of the lan gene cluster. The plasmid can be used for deletion of the entire lan gene cluster in B. lichenformis SJ1904 derivatives.

### Molecular Biological Methods

DNA manipulations and transformations were performed by standard molecular biology methods as described in: Sambrook et al. (1989): Molecular cloning: A laboratory manual. Cold Spring Harbor laboratory, Cold Spring Harbor, N.Y. Ausubel et al. (eds) (1995): Current protocols in Molecular Biology. John Wiley and Sons. Harwood and Cutting (eds) (1990): Molecular Biological Methods for Bacillus. John Wiley and Sons.

Enzymes for DNA manipulation were obtained from New England Biolabs, Inc. and used essentially as recommended by the supplier.

Competent cells and transformation of B. subtilis was obtained as described in Yasbin et al. (1975): Transformation and transfection in lysogenic strains of Bacillus subtilis: evidence for selective induction of prophage in competent cells. J. Bacteriol. 121, 296-304.

Conjugation of B. licheniformis was performed as described in several patents (U.S. Pat. No. 5,695,976A, 5,733,753A, 5,843,720A, 5,882,888A, WO2006042548A1)

### Standard Cultivation Procedure

All growth media were sterilized by methods known in the art. Unless otherwise described, tap water was used. The ingredient concentrations referred to in the below recipes are before any inoculation.

First inoculum medium: SSB4 agar. Soy peptone SE50MK (DMV) 10 g/l; sucrose 10 g/l; Di-Sodiumhydrogenphosphate, 2H₂0 5 g/l; Potassiumdihydrogenphosphate 2 g/l; Citric acid 0.2 g/l; Vitamins (Thiamin-hydrochlorid 11.4 mg/l; Riboflavin 0.95 mg/l; Nicotinic amide 7.8 mg/l; Calcium D-pantothenate 9.5 mg/l; Pyridoxal-HCl 1.9 mg/l; D-biotin 0.38 mg/l; Folic acid 2.9 mg/l); Trace metals (MnS04, H₂0 9.8 mg/l; FeS04, 7H₂0 39.3 mg/l; CuS04, 5H₂0 3.9 mg/l; ZnS04, 7H₂0 8.2 mg/l); Agar 25 g/l. Use of deionized water. pH adjusted to pH 7.3 to 7.4 with NaOH.

Transfer buffer. M-9 buffer (deionized water is used): Di-Sodiumhydrogenphosphate, 2H₂0 8.8 g/l; Potassiumdihydrogenphosphate 3 g/l; Sodium Chloride 4 g/l; Magnesium sulphate, 7H₂0 0.2 g/l.

Inoculum shake flask medium (concentration is before inoculation): PRK-50: 1 10 g/l soy grits; Di-Sodiumhydrogenphosphate, 2H₂0 5 g/l; pH adjusted to 8.0 with NaOH/H3P04 before sterilization.

Make-up medium (concentration is before inoculation): Tryptone (Casein hydrolysate from Difco) 30 g/l; Magnesium sulphate, 7H₂0 4 g/l; Di-Potassiumhydrogenphosphate 7 g/l; Di-Sodiumhydrogenphosphate, 2H₂0 7 g/l; Di-Ammoniumsulphate 4 g/l; Potassiumsulphate 5 g/l; Citric acid 0.78 g/l; Vitamins (Thiamin-hydrochlorid 34.2 mg/l; Riboflavin 2.8 mg/l; Nicotinic amide 23.3 mg/l; Calcium D-pantothenate 28.4 mg/l; Pyridoxal-HCl 5.7 mg/l; D-biotin 1.1 mg/l; Folic acid 2.5 mg/l); Trace metals (MnS04, H₂0 39.2 mg/l; FeS04, 7H₂0 157 mg/l; CuS04, 5H₂0 15.6 mg/l; ZnS04, 7H₂0 32.8 mg/l); Antifoam (SB2121) 1.25 ml/l; pH adjusted to 6.0 with NaOH/H3PO4 before sterilization.

Feed medium: Sucrose 708 g/l;

Inoculum steps: First the strain was grown on SSB-4 agar slants 1 day at 37° C. The agar was then washed with M-9 buffer, and the optical density (OD) at 650 nm of the resulting cell suspension was measured. The inoculum shake flask (PRK-50) was inoculated with an inoculum of OD (650 nm)×ml cell suspension=0.1. The shake flask was incubated at 37° C. at 300 rpm for 20 hr. The fermentation in the main fermentor (fermentation tank) was started by inoculating the main fermentor with the growing culture from the shake flask. The inoculated volume was 11% of the make-up medium (80 ml for 720 ml make-up media).

Standard lab fermentors were used equipped with a temperature control system, pH control with ammonia water and phosphoric acid, dissolved oxygen electrode to measure oxygen saturation through the entire fermentation.

Fermentation parameters: Temperature: 38° C.; The pH was kept between 6.8 and 7.2 using ammonia water and phosphoric acid; Control: 6.8 (ammonia water); 7.2 phosphoric acid; Aeration: 1.5 liter/min/kg broth weight.

Agitation: 1500 rpm.

Feed strategy: 0 hr. 0.05 g/min/kg initial broth after inoculation; 8 hr. 0.156 g/min/kg initial broth after inoculation; End 0.156 g/min/kg initial broth after inoculation.

Experimental setup: The cultivation was run for five days with constant agitation, and the oxygen tension was followed on-line in this period. The different strains were compared side by side.

### Example 1: B. licheniformis SJ12173 Expressing Alkaline Protease AprH

A B. licheniformis host strain expressing six site-specific chromosomally integrated copies of an AprH expression construct was constructed using standard methods, for example as described in U.S. Pat. Nos. 5,695,976, 5,733,753, 5,843,720, 5,882,888 and/or WO2006042548. The expression construct encoded the aprL signal peptide from Bacillus licheniformis in translational fusion with the aprH pro-peptide and mature peptide from Bacillus clausii (shown in SEQ ID NO:39) The recipient host was a B. licheniformis SJ1904 derivative (WO2008066931). The resulting six-copy AprH expression host was denoted SJ12713.

### Example 2: Temperature-Sensitive Deletion-Plasmid for B. licheniformis lanA1

Plasmid pBKQ1697 was designed to delete the structural lanA1 gene within the B. licheniformis lan gene cluster.

Colony PCR was performed on B. licheniformis SJ1904. A first 1.1 kb fragment of the B. licheniformis SJ1904 chromosome, containing the upstream region of lanA1, was amplified by PCR using primers pr535 and pr536 by standard PCR. A cleavage site for restriction enzyme MluI was incorporated into primer pr535. A cleavage site for restriction enzyme BamHI was incorporated into primer pr536.

A second 1.1 kb fragment of the B. licheniformis SJ1904 chromosome, containing the flanking region immediate downstream of lanA1, was PCR amplified using primers pr537 and pr538. A cleavage site for the BamHI restriction enzyme (bold) was incorporated into primer pr537. A cleavage site for the SacII restriction enzyme (bold) was incorporated into primer pr538.

The resulting two DNA fragments were amplified by PCR using the PHUSION HOT START® II DNA polymerase (Thermo Fisher Scientific). The PCR amplification reaction mixture contained B. licheniformis SJ1904 genomic DNA (10 μl template solution (colony solution cooked at 99 C for 10 minutes in H₂O), 1 μl of sense primer (20 pmol/μl), 1 μl of anti-sense primer (20 pmol/μl), 10 μl of 5× PCR buffer, 8 μl of dNTP mix (5 mM each), 18.5 μl H₂O, and 0.5 μl (2 U/μl) DNA polymerase mix. An Eppendorf Mastercycler thermocycler was used to amplify the fragment with the following settings: One cycle at 94° C. for 2 minutes; 25 cycles each at 94° C. for 30 seconds, 54° C. for 45 seconds, 72° C. for 60 seconds; one cycle at 72° C. for 5 minutes; and 10° C. hold. The PCR products were purified from a 1% agarose SYBR® Safe DNA gel stain gel (Life Technologies) with 0.5× TBE buffer using the Qiagen QIAquick Gel Extraction Kit (Qiagen, Inc., Valencia, Calif.) according to the manufacturer's instructions.

The two purified PCR products were digested with restriction enzyme BamHI as follows: 45 μl purified PCR, 5 μl NEB2 buffer, 1 μl BamHI and incubated for 1 hour at 37° C. The digested DNA was subsequently purified using Qiagen PCR purification kit according to manufacturer's instructions. The two PCR products were mixed and ligated as follows: 4.25 μl of each digested PCR product, 1 μl 10× Ligation buffer and 0.5 μl T4 DNA ligase. Ligation mixture was incubated at room temperature for 1 hour.

A subsequent PCR amplification using the ligated PCR fragments as template DNA was performed to create a single fragment using the PHUSION HOT START® II DNA polymerase (Thermo Fisher Scientific) as follows: The PCR amplification reaction mixture contained 10 μl of a 100 times diluted ligation mixture described above, 1 μl of primer pr535 (20 pmol/μl), 1 μl of primer pr538 (20 pmol/μl), 10 μl of 5× PCR buffer, 8 μl of dNTP mix (5 mM each), 18.5 μl H₂O, and 0.5 μl (2 U/μl) PHUSION HOT START® II DNA polymerase (Thermo Fisher Scientific). An Eppendorf Mastercycler thermocycler was used to amplify the fragment with the following settings: One cycle at 94° C. for 2 minutes; 25 cycles each at 94° C. for 30 seconds, 54° C. for 45 seconds, 72° C. for 3 minutes; one cycle at 72° C. for 5 minutes; and 10° C. hold, resulting in a 2.2 kb PCR fragment.

The resulting PCR product (lig-PCR lanA1 flanks; SEQ ID NO:40) containing the flanking upstream and downstream region of lanA1 ligated in the BamHI site was run on a 1% agarose TBE gel and purified on Qiagen QIAquick Gel Extraction Kit according to manufacturer's instructions. The purified PCR product was subsequently digested with MluI and SacII as follows: 45 μl purified PCR product, 5 μl NEB2 buffer, 1 μl MluI, and 1 μl SacII and incubated at 37° C., resulting in a 2.2 kb fragment. In another tube, plasmid vector pPP3932 was digested with MluI and SacII according to manufacturer's instructions, resulting in a 5.7 kb fragment.

The digested PCR product and plasmid were subsequently run on a 1% agarose gel by electrophoresis using TBE buffer followed by purification using the Qiagen QIAquick Gel Extraction Kit (Qiagen, Inc.) according to manufacturer's instructions. The purified DNA fragments were then ligated using T4 DNA ligase as follows: 1 μl pPP3932 fragment, 1 μl PCR product, 6.5 μl H₂O, 1 μl ×10 T4 DNA ligase buffer, 0.5 μl T4 DNA ligase. The ligase reaction was incubated at room temperature for 2 hours. The 10 μl aliquot of the ligation was used to transform E. coli TG1 cells according to the manufacturer's instructions.

Plasmid DNA was prepared from E. coli transformants and confirmed by restriction analysis and subsequent sequencing with primers: pr535, pr536, pr537, pr538, pr539, pr540, pr541, and pr542.

The verified plasmid was then used to transform donor strain B. subtilis PP3724 as described previously in Materials and Methods, resulting in B. subtilis BKQ1707. Donor strain B. subtilis BKQ1707 was subsequently used for conjugation of B. licheniformis SJ1904 derivatives according to method described above in order to introduce the temperature sensitive plasmid to the relevant strains.

### Example 3: Temperature-Sensitive Deletion-Plasmid for B. licheniformis lan Gene Cluster

Plasmid pBKQ1751 was designed to delete the entire lan gene cluster (SEQ ID NO:41) in B. licheniformis. Colony PCR was performed on B. licheniformis SJ1904. A 1.05 kb fragment of the B. licheniformis SJ1904 chromosome, containing the upstream region of the lan gene cluster, was amplified by PCR using primers pr547 and pr548 by standard PCR. A cleavage site for restriction enzyme MluI was incorporated into primer pr547. A cleavage site for restriction enzyme BamHI was incorporated into primer pr548.

A second 1.05 kb fragment of the B. licheniformis SJ1904 chromosome, containing the flanking region immediate downstream to the lan gene cluster, was amplified by PCR by standard PCR technique using primers pr549 and pr550. A cleavage site for the BamHI restriction enzyme (bold) was incorporated into primer pr549. A cleavage site for the MluI restriction enzyme (bold) was incorporated into primer pr550.

The respective DNA fragments were amplified by PCR using the PHUSION HOT START® II DNA polymerase (Thermo Fisher Scientific) (Thermo Scientific). The PCR amplification reaction mixture contained B. licheniformis SJ1904 genomic DNA (10 μl template solution (colony solution cooked at 99° C. for 10 minutes in H₂O), 1 μl of sense primer (20 pmol/μl), 1 μl of anti-sense primer (20 pmol/μl), 10 μl of 5× PCR buffer, 8 μl of dNTP mix (5 mM each), 18.5 μl H₂O, and 0.5 μl (2 U/μl) DNA polymerase mix.

An Eppendorf Mastercycler thermocycler was used to amplify the fragment with the following settings: One cycle at 94° C. for 2 minutes; 25 cycles each at 94° C. for 30 seconds, 54° C. for 45 seconds, 72° C. for 60 seconds; one cycle at 72° C. for 5 minutes; and 10° C. hold. The PCR products were purified from a 1% agarose SYBR® Safe DNA gel stain gel (Life Technologies) with 0.5× TBE buffer using the Qiagen QIAquick Gel Extraction Kit (Qiagen, Inc., Valencia, Calif.) according to the manufacturer's instructions.

The two purified PCR products were digested with restriction enzyme BamHI as follows: 45 μl purified PCR, 5 μl NEB2 buffer, 1 μl BamHI and incubated for 1 hour at 37 C. The digested DNA was subsequently purified using Qiagen PCR purification kit according to manufacturer's instructions.

The two PCR products were mixed and ligated as follows: 4.25 μl of each digested PCR product, 1 μl 10× Ligation buffer and 0.5 μl T4 DNA ligase. Ligation mixture was incubated at room temperature for 1 hour. A subsequent PCR amplification using the ligated PCR fragments as template DNA was performed to create a single fragment using the PHUSION HOT START® II DNA polymerase (Thermo Fisher Scientific) as follows: The PCR amplification reaction mixture contained 10 μl of a 100 times diluted ligation mixture described above, 1 μl of primer pr535 (20 pmol/μl), 1 μl of primer pr538 (20 pmol/μl), 10 μl of 5× PCR buffer, 8 μl of dNTP mix (5 mM each), 18.5 μl H₂O, and 0.5 μl (2 U/μl) PHUSION HOT START® II DNA polymerase (Thermo Fisher Scientific).

An Eppendorf Mastercycler thermocycler was used to amplify the fragment with the following settings: One cycle at 94° C. for 2 minutes; 25 cycles each at 94° C. for 30 seconds, 54° C. for 45 seconds, 72° C. for 3 minutes; one cycle at 72° C. for 5 minutes; and 10° C. hold, resulting in a 2.1 kb PCR fragment.

The resulting PCR product (lig-PCR lan gene cluster flanks; SEQ ID NO:42) containing the flanking upstream and downstream region of the entire lan gene cluster was run on a 1% agarose TBE gel and purified on Qiagen QIAquick Gel Extraction Kit according to manufacturer's instructions. The purified PCR product was subsequently digested with MluI as follows: 45 μl purified PCR product, 5 μl NEB3 buffer and 1 μl MluI and incubated at 37° C., resulting in a 2.1 kb fragment.

In another tube, plasmid vector pPP3932 was digested with MluI and treated with Calf Intestine Phosphatase according to manufacturer's instructions, resulting in a 5.8 kb fragment.

The digested PCR product and plasmid were subsequently run on a 1% agarose gel by electrophoresis using TBE buffer followed by purification using the Qiagen QIAquick Gel Extraction Kit (Qiagen, Inc.) according to manufacturer's instructions.

The purified DNA fragments were then ligated using T4 DNA ligase as follows: 2 μl pPP3932 fragment, 0.5 μl PCR product, 5 μl H₂O, 1 μl ×10 T4 DNA ligase buffer, 0.5 μl T4 DNA ligase. The ligase reaction was incubated at room temperature for 2 hours. The 10 μl aliquot of the ligation was used to transform 50 μl E. coli TG1 cells according to the manufacturer's instructions. Plasmid DNA was prepared from E. coli transformants and confirmed by restriction analysis and subsequent sequencing with primer pr547, pr548, pr549, pr550, pr551, pr552, pr553 and pr554.

In order to enable deletion of the entire lan gene cluster (approximately 15.2 kb), a chloramphenicol resistance gene surrounded by resolvase recognizable regions (res-sites) was inserted between the upstream and downstream flanking regions of the lan gene cluster present in pBKQ1699 as follows: Plasmid pSJ3372, which contains a res-cat-res region (see U.S. Pat. No. 5,882,888) surrounded by a BclI and a BamHI site, was digested with BclI and BamHI according to manufacturer's instructions, resulting in a 1.2 kb fragment containing the res-cat-res region.

Plasmid pBKQ1699 was digested with BamHI and treated with Calf Intestine Phosphatase by standard technique, resulting in a 7.9 kb fragment. The digestion mixtures were run on 1% agarose gel by electrophoresis using TBE buffer followed by purification using the Qiagen QIAquick Gel Extraction Kit (Qiagen, Inc.) according to manufacturer's instructions.

The purified DNA fragments were then ligated using T4 DNA ligase as follows: 3 μl pBKQ1699 fragment (plasmid vector), 0.5 μl pSJ3372 fragment (res-cat-res), 5 μl H₂O, 1 μl ×10 T4 DNA ligase buffer, 0.5 μl T4 DNA ligase. The ligase reaction was incubated overnight at 16° C. The 10 μl aliquot of the ligation was used to transform 50 μl E. coli TG1 cells according to the manufacturer's instructions. Plasmid DNA was prepared from E. coli transformants and confirmed by restriction analysis, resulting in pBKQ1751 in which the res-cat-res region was inserted in between the flanking regions of the lan gene cluster in pBKQ1699.

The verified plasmid pBKQ1751 was then used to transform donor strain B. subtilis PP3724 as described previously in Materials and Methods, resulting B. subtilis BKQ1754. Donor strain B. subtilis BKQ1754 was subsequently used for conjugation of B. licheniformis SJ1904 derivatives according to method described above in order to introduce the temperature sensitive plasmid to the relevant strains.

### Example 4: Deletion of lanA1 in B. licheniformis

Donor strain B. subtilis BKQ1707 was used for conjugation of B. licheniformis recipients as previously described (U.S. Pat. No. 5,843,720) in order to introduce the temperature sensitive plasmid pBKQ1697 to the relevant strains.

B. licheniformis conjugants containing plasmid pBKQ1697 were then grown on LB PGS selective medium at 50° C. to force integration of the vector. Selection of strains with chromosomal integration of the plasmid was performed based on their ability to grow on LB PGS+5 microgram/ml of erythromycin at 50° C. These strains were then grown without selection on LB PGS plates at 34° C. to allow excision of the integrated plasmid.

A streak of culture was inoculated in 10 ml LB medium and incubated for 6 hours at 34° C. Dilution series were made in LB medium and the diluted cell cultures were plated on LB PGS plates and incubated overnight at 37° C. Next day, replica plating was performed on LB PGS and LB PGS+5 microgram/ml of erythromycin. The plates were incubated overnight at 34° C.

Next day, erythromycin sensitive colonies were identified. Colony PCR on a series of erythromycin sensitive colonies was performed with primer pr601 and primer pr602 in order to identify strains in which lanA1 has been deleted.

Using temperature sensitive plasmid pBKQ1697 for deletion of lanA1 in B. licheniformis SJ1904 derivatives by homologeous recombination, the following strain was isolated: B. licheniformis BKQ1944 (AprH producing).

### Example 5: Deletion of the Entire lan Gene Cluster in B. licheniformis

Donor strain B. subtilis BKQ1754 was used for conjugation of B. licheniformis recipients as previously described (U.S. Pat. No. 5,843,720) in order to introduce the temperature sensitive plasmid pBKQ1751. B. licheniformis conjugants containing plasmid pBKQ1751 were then grown on LB PGS plates supplemented with 6 microgram/ml of chloramphenicol and incubated at 50° C. to force integration of the plasmid.

Strains with chromosomal integrated plasmids were selected based on their ability to grow on LB PGS+6 microgram/ml of chloramphenicol at 50° C. The selected strains were then re-streaked on LB PGS plates supplemented with 6 microgram/ml of chloramphenicol and incubated at 34° C. to allow excision of the integrated plasmid.

Next day, a streak of culture was inoculated in 10 ml LB medium supplemented with 6 microgram/ml of chloramphenicol and incubated for 6 hours at 34° C. Dilution series were made in LB medium and the diluted cell cultures were plated on LB PGS+6 microgram/ml chloramphenicol and incubated overnight at 37° C.

Next day, replica plating was performed on LB PGS+6 microgram/ml chloramphenicol and LB PGS+5 microgram/ml erythromycin. The plates were incubated overnight at 34° C. Next day, erythromycin sensitive colonies were identified. Colony PCR on a series of erythromycin sensitive colonies was performed with primer pr555 and primer pr556 in order to identify strains in which the entire lan gene cluster (approximately 15.2 kb has been deleted and replaced by a res-cat-res region.

Using temperature sensitive plasmid pBKQ751 for deletion of the entire lan gene cluster in B. licheniformis SJ1904 derivatives by homologeous recombination, the following strain was isolated: B. licheniformis BKQ1946 (AprH producing).

### Example 6. AprH in B. licheniformis Strains with lanA1 or lan Gene Cluster Deleted

Four independent cultures of each of AprH-producing B. licheniformis SJ12713 (reference), B. licheniformis BKQ1944 (ΔlanA1) and B. licheniformis BKQ1946 (Δlan gene cluster) were cultivated. Samples were regularly taken once a day for a period of five days. The titer and yield of AprH were then measured. After day 5, significantly increased AprH titers and yields were found in both the strain with a deleted lanA1 and in the strain with a deleted lan gene cluster, when compared with the reference strain B. licheniformis SJ12713. The results are listed in table 2 below. The data clearly show that deletion of lanA1 or the entire lan gene cluster results in significantly increased yields of AprH when compared to the control reference strain.

A similar expression study of the AmyL amylase in a 4 gene copy lan gene cluster deleted host strain was carried out which demonstrated yield improvements in a lan gene cluster deleted host strain of about 2% compared with the control reference strain (data not shown).

TABLE 2
Relative titer and total yield in protease
AprH producing B. licheniformis strains.

| | | Relative | STDEV | Relative | STDEV |
|---|---|---|---|---|---|
| Strain | Deletion | Titer % | (Titer) % | Total yield % | (Yield) % |
| SJ12713 | control | 100 | 3.8 | 100 | 4.7 |
| BKQ1944 | IanA1 | 104.8 | 3.0 | 106.3 | 1.6 |
| BKQ1946 | Ian gene | 104.9 | 1.9 | 106.3 | 3.4 |
| | cluster | | | | |

1-10. (canceled)

11. A Bacillus licheniformis host cell producing a heterologous polypeptide of interest encoded by an exogenous polynucleotide integrated into the chromosome of the host cell in at least one copy, wherein at least one gene in the lan gene cluster is inactivated by a non-sense mutation in said at least one gene, a partial deletion of said at least one gene or a full deletion of said at least one gene.

12. The host cell of claim 11, wherein the polypeptide of interest is expressed with or without a secretion signal peptide.

13. The host cell of claim 11, wherein the polypeptide of interest is an enzyme.

14. The host cell of claim 13, wherein the enzyme is an oxidoreductase, transferase, hydrolase, lyase, isomerase, or ligase; preferably the enzyme is an aminopeptidase, amylase, asparaginase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, hyaluronic acid synthase, invertase, laccase, lipase, mannosidase, mutanase, oxidase, a pectinolytic enzyme, peroxidase, phytase, polyphenoloxidase, protease, ribonuclease, transglutaminase, or xylanase.

15. The host cell of claim 11, wherein the at least one gene in the lan gene cluster is selected from the group consisting of a lanI gene having a nucleotide sequence at least 70% identical to the lanI shown in SEQ ID NO:1, a lanH gene having a nucleotide sequence at least 70% identical to the lanH shown in SEQ ID NO:2, a lanE gene having a nucleotide sequence at least 70% identical to the lanE shown in SEQ ID NO:3, a lanG gene having a nucleotide sequence at least 70% identical to the lanG shown in SEQ ID NO:4, a lanF gene having a nucleotide sequence at least 70% identical to the lanF shown in SEQ ID NO:5, a lanY gene having a nucleotide sequence at least 70% identical to the lanY shown in SEQ ID NO:6, a lanR gene having a nucleotide sequence at least 70% identical to the lanR shown in SEQ ID NO:7, a lanX gene having a nucleotide sequence at least 70% identical to the lanX shown in SEQ ID NO:8, a lanP gene having a nucleotide sequence at least 70% identical to the lanP shown in SEQ ID NO:9, a lanT gene having a nucleotide sequence at least 70% identical to the lanT shown in SEQ ID NO:10, a lanM2 gene having a nucleotide sequence at least 70% identical to the lanM2 shown in SEQ ID NO:11, a lanA2 gene having a nucleotide sequence at least 70% identical to the lanA2 shown in SEQ ID NO:12, a lanA1 gene having a nucleotide sequence at least 70% identical to the lanA1 shown in SEQ ID NO:13 and a lanM1 gene having a nucleotide sequence at least 70% identical to the lanM1 shown in SEQ ID NO:14.

16. The host cell of claim 11, wherein the at least one gene in the lan gene cluster is selected from the group consisting of a lanI gene having the nucleotide sequence shown in SEQ ID NO:1, a lanH gene having the nucleotide sequence shown in SEQ ID NO:2, a lanE gene having the nucleotide sequence shown in SEQ ID NO:3, a lanG gene having the nucleotide sequence shown in SEQ ID NO:4, a lanF gene having the nucleotide sequence shown in SEQ ID NO:5, a lanY gene having the nucleotide sequence shown in SEQ ID NO:6, a lanR gene having the nucleotide sequence shown in SEQ ID NO:7, a lanX gene having the nucleotide sequence shown in SEQ ID NO:8, a lanP gene having the nucleotide sequence shown in SEQ ID NO:9, a lanT gene having the nucleotide sequence shown in SEQ ID NO:10, a lanM2 gene having the nucleotide sequence shown in SEQ ID NO:11, a lanA2 gene having the nucleotide sequence shown in SEQ ID NO:12, a lanA1 gene having the nucleotide sequence shown in SEQ ID NO:13 and a lanM1 gene having the nucleotide sequence shown in SEQ ID NO:14.

17. The host cell of claim 11, wherein two or more genes in the lan gene cluster are inactivated; preferably three or more genes in the lan gene cluster are inactivated; even more preferably four, five, six, seven, eight, nine, ten, eleven, twelve or thirteen or more genes in the lan gene cluster are inactivated.

18. The host cell of claim 7, wherein the genes in the lan gene cluster are inactivated by a non-sense mutation, a partial deletion or a full deletion of said genes, or by a combination thereof.

19. The host cell of claim 11, wherein the entire lan gene cluster is deleted.

20. A method for producing a polypeptide of interest, said method comprising:
a) cultivating the Bacillus licheniformis host cell of claim 11 in a medium and under conditions conducive for the production of said polypeptide; and optionally
b) recovering said polypeptide.


SEQUENCE LISTING (42 sequences)

SEQ ID NO: 1 - DNA, 942 bp, Bacillus licheniformis
  gene (1)..(942): lanI open reading frame of B. licheniformis
atgagggtcttgaaaaatgaactttacaggctgatggtgacgaaaagtacctggattgtgttaagcttgctgcttgtcatgacaatcgctgttgcatggatggtcagcaatggcgaaaaggagaaggagacaggtaactggaaagagcaattaaccgttcaaaacgctcagtatgaaagagaaatgagagagctgagcccagcggttcccaaataccaatttttaaaagaagagatcgcggtcaatcaataccggcttgagcataatttgccgccttctgcgaaatacaatgtttggacgatgctgaaggagttaaaaccgatcacaacattaatcgcgttgattgcaatcgtgctggcagcgaactccatcgcgcttgagcacagcaaaggaactattaaatttgcgattgcaacaccggtcaagcgttggcattatctattggggaaatacttgtcgatcttgctcaacactgtatttatgtttgctgcgacacttttatttgcgtttgttttagggtatgccctgctaggtttggagggaagccaatactatttgtcctatcgaagcggcgaagtgataaagatgtcaatgctgaagtttttagccttagactatggagccgctttattgaatattattgtgctggccacgctggcctttatgatttcggtcatcctaagaagcgcggtcgtctctgtcggtctttcgctgttcgtcttttttacagggtctgcgattactcaatttttagcggcaaaatttgattggaccaagtatacgatctttgcaaattcagatctcagtcaatacattgatggggagccgtttattcaagatatgactctttcattttcagcggctgtgattgctgtttattttattctttttctggctgtctccttttgggtttttcaaaagcgggatattgtcacgtcataa

SEQ ID NO: 2 - DNA, 903 bp, Bacillus licheniformis
  gene (1)..(903): lanH open reading frame of B. licheniformis
atgagtcaagtcgaatttgaaggtgtaagtaaacgaataaaaggcagaccaattgtccaaaatatcacatttcaaattgccccaggtacaatttttgggctgctcgggccaaacggcgctggcaagacaacacttatcaaaatgattgtcgggatggcaaagccgacatcaggagatatccgcatcgacggctattcagttaaaagcaattacgaggaagcggcagcccgagtcggttctgttgttgaaaacccatccttttatgagcacttaacaggataccaaaaccttaaatatctcggcggattccacagccacgtgtcaaaggagcgcatagaagagatcgttcagcttgttgatttgacaggaagtattcataaaccagttaaaacgtattcattaggcatgaaacagcgtttgggccttgccgtcgcgctcttgcatgatccggaatttctcattctcgatgaaccgacaaacggccttgatcctcagggaatcattgatttgcgcgaacaccttcagtacttggcgaaaaccttcaacaaaacgattttgatttcgagtcatcttctgtctgaggttgagatgatttgtgatgaatacggcgtcatgaaaaacggagaactcctgcaaattaagagcaatcaccgcgataccgatacggttcgttatcggcttacattaaacggccacgccgatgaagcggctgacctgttgaatgagtaccagtatgcaggcggtctcacggaagataaaaatgagatttatgtcctttgcatggaagaagacattatgaaagtcgttaatctgttaatggagaacaaaataagagttctgcatatgaagcaggaaaaacagtcgatagaacaaagctttctggaattgatcaataaggggtga

SEQ ID NO: 3 - DNA, 750 bp, Bacillus licheniformis
  gene (1)..(750): lanE open reading frame of B. licheniformis
gtgaaaaatttgctcaaagcggaatggatcaaactcaggaactctgattttacaatgacgagcatattcattcttaccgcatctttagtgtttacagggtttatggcaaaacgaatcaccccgtttgcgggagggaatgaatggacatcgttaaggcatcttacattgctgttggtattcagcacgatttatccctggcttgtctcttcggtcacagtatttttgcataaggatgaactgaaaaaccgagtgtggctgaatcgaatcttatcgccgcagcctttcgcagccatgctttttgctaaggttttgtttattggactgttcgcagccgcattatatgccgtctgtgtgatcgaatcgcttgccttcggtttaatatcggggttccccgggcctgccccttggttttcttggatgctgggcttcattctcaatctcttgtcaacctttccgctgatctgtacggtcctatggcttaaattcaccagtaaggaaaagacctcattaaacattatcgtattcatttgttcactgctgagtttcggcgtttcgcaatcccctttagggcttctctttccatggtcttttcctgtgacggccctcattgcaatggagaagtcagttgccgttcttatcggttcgattgtttggttcacggcggtttcgctgctctttttttctcttctattgaaacagatcaggccataccaaaaaggaggaatacaaaatgagtcaagtcgaatttga

SEQ ID NO: 4 - DNA, 714 bp, Bacillus licheniformis
  gene (1)..(714): lanG open reading frame of B. licheniformis
atgaaattgtttaaagtttatttttggttaagctgtcccgaagcggtgaaaaagagaatgaaatttcagacgtttgcatgtacggttatgatcatcacagtgctcgccgttttgcttaattatcttctcttttataaaaacggggcaaataaagaaatccgtagcatggagggaatgactgtttatgtcgtctcggtttggctgacttatattacaggcagaagattatttggacgcatgatgcatcaagcttttatcttgcggtatcccgttcgcctgttcacttgggtgctgactggaatcgccgtgataactatcgtctcagcgctggtcttgctttacggctgtttggcagtcagtctgctgtttcaaaaggatgttttgttttcgccagagcgtttagccgctgttgcgatactggcggtgatcatgtcttccgtccagttttttctgaagacgttaaaggcagaatatctatttgtgctgttgatgacgctgacgccgttttttatgcaaagtggaacactttatttgccgtggggagtttcttggcttttgttgactgaatattcagtgaacgatataggcctggcagtttatcttatttcggcgggttatatagcggtctgtatgtgttggatcttaaggagcggggaggaaaagatccgtgaaaaatttgctcaaagcggaatggatcaaactcaggaactctga

SEQ ID NO: 5 - DNA, 636 bp, Bacillus licheniformis
  gene (1)..(636): lanF open reading frame of B. licheniformis
atggttgagcttccaattgtcatgaaacatgttgaggtgcgggcgggagacaggaaacttcttgaaaacgtcagccttgctgtggacaaagggagccgggttttgctcaaaggagaaaacggatcgggaaaatcgacgtttcttaaagcaattaccggcttgtctttatttgcgggcgaaattttgatttacggtcaatcgattatccaaaacagggaagctgctgtcgggcatatcggctatgtcagtgacgaagtgccgcttcatgagcatcttactggaacggacaatctgagagtccatgccttgcttcacggaatagatgacgatgaccggatctattctgaactagagcggttcggtatcaatccggctgatgacacgaaagtacagtactattcaacaggcatgaggcaaaagctgaagatcgcaatggccttatttcatcagccttccatcttaatattggatgagcctttaaatgggctcgaccgaaaagcgcaggaaagcgttgtagacatcataaggcggtatgacgggaccatcatggcatcaagccattggaacgaaacttttatcgaattgtttgatcgttttctagccatagaaagcgggcgtttaaatgaaattgtttaa

SEQ ID NO: 6 - DNA, 288 bp, Bacillus licheniformis
  gene (1)..(288): lanY open reading frame of B. licheniformis
atgctagaaatgatcaattcaattggtgctgtcgcaggatttgcggggtttatcggaatcattattttaatcagccttgacggaaaggatgaacgcggggcatatattcaaaacaaattctttcgggtgatgttctttttgcttacacttggaatatccgccgttatttttacaagttcatgggtcgatttgtcgttcgccaattataaaaacatggtcacactggctttttcgctgccgtatttcattggattcttcattttgttgggaattcgttcaagggtgtag

SEQ ID NO: 7 - DNA, 279 bp, Bacillus licheniformis
  gene (1)..(279): lanR open reading frame of B. licheniformis
ttgtactctatatatgtaaatgatttgcaaataaagagggtgattgagattgaaaagcaaagcggggtgttaaccaaccggattcctgtgctgcgagcagagaaagggtggacgcaaaagcagcttgccgatctattgggtgtaacaagacaaactgtgatttccattgaaaagaataaatacaaaccatccctgttaatggggtttcgcattgccgcgttatttgagaaggacatcaacgaggtatttcaataccaactagaggaggatgatcgctga

SEQ ID NO: 8 - DNA, 189 bp, Bacillus licheniformis
  gene (1)..(189): lanX open reading frame of B. licheniformis
atgtatgaatatcaaacagtttcaatcgctgtaggagcattcaacggaaaacttgagaaaaattattctgatgtgattcatgaatatgctgaaaaaggctggaagcttcacagcgtcttttctgttccatcaagggccggaggccaggtttcttctatcgaactgatttttgaaaaacctaaatcttga

SEQ ID NO: 9 - DNA, 1332 bp, Bacillus licheniformis
  gene (1)..(1332): lanP open reading frame of B. licheniformis
gtgaaaagaatatatatttttctcttatgttttgcagtcctgctgccggtcggcggaaagacggctcaagcaaaagaacaagcaggagaacagtatcttttgcttgaacatgtaaaagataaatcgaaactgctggacacggcggaacaatttcacatccatgccgatgtcattgaagaaatcggacttgcaaaagtgaccggtgaaaaacaaaagcttgctccttttacaaagaagcttgctgaaaaagtcggggctgacgtcattgaaaagccgattgcaaatacagcagtaaacgaaacggaatcagtcatcagcggttcgcctgcatgggggcttgacggtattttggaactaaaagaatatctatggttcgctgcaaagcagacggattcctatcgcacgtatcaaatcgaaagagggcatccggacgtcaaagttgccttgattgacagcggactggatcttgaccatccggacttaaaagcgtccgtcaatacaaatggcggctggaattacattgacggcaagcctgtatccggagatccgacaggacacggaacacaaacagccgggatgatcaatatcattgcgccagatgtgacgataacgccttatcaagtgctggacgaaaaaggcggagacagctacaacatcatgaaggcgatggttgatgcagtcaatgacgggcatgaagtcatcaacatcagtacgggaagctatacttctcttgacagggaaggaaaagtgctgatgaaagcttatcaaagggctgcaaactacgctgcaaagcatcaggtgctggtcttctcttccgccggcaataaaggagtcaaccttgatgagatgaggaaaacggaaaacaaggtgcatttgccgagcgctttgaagcacgtcgtatcagtcagcagcaatatgaaaagcaacaatatttccccatactccaatcaagggcgggaaattgaattcacagccccgggaggatatttgggagaaacgtatgatcaggatgggatggttcgtgtgacagacctcgtactgacaacgtatccgaaagggaaggacaacaccgcattagaccagatgctaaacatcccaaagggatattccctctcatacggaacaagcttggccgccccgcaggtagctggaacagctgcactggttatatctgaataccgggagcgccatcacaggaagccatcggctaaacaagtgcatcacatcctgaggaaatcggcattagacctgggcaaaccggggaaagatgttatatacggctacggggaagtacgcgcttatcaagcgctgaaaatgatgaacaaggagtga

SEQ ID NO: 10 - DNA, 2157 bp, Bacillus licheniformis
  gene (1)..(2157): lanT open reading frame of B. licheniformis
ttgttttttcataagacaccgtttatagaacagatgcagcagacggaatgcggactgtgctgtatggcgatggtagccggacggtatggttcgcatcatactctgcatgagctgagggatttatcgggatgcggcagggacggtatgaccctttttcatatgcgtcagctcagcgaaaatctgggatttgacgcaaaggtgtacagggcaggcgcggcagaacttccacatgtgcgtctgccggcaatcgctttttggtccgataatcattatgtggttattgatcaaataaaagaccggcatgtcaacatcatcgatcctgcgattggacgaaaaaaactcagtatcgaagcttttctcgaaaattacagcgggatcgtgctggagatgatccctaccgagcggatcaggccgaagaaaaaaccgcccgtctggaggcattttctttttcatttaaaagaatccccttctctgcttgcgaccgtgctgttgtttgcgatgatctttcagctcgtttcattaggcataccgatgctcgtgcaatacctgatcgatgacatccttgctcaaaatcagcaggctctgctgcaaacatttatgacgggcgttcttgttttaatgctcgttcaaggcacggttcaattgatcaggggtcaatttatcattaaattaaataacttccttgacaaacgaatgatgagaacatttttctcgcatattctgaagcttccctatcaattctttcaattgaggtcgttcggggatctccttttccgcgcgacaagcctccggattgtcagggatatgatgtcttcccaattggtcctcggcgtactcgattttggcgcacttttatttatctcgttttatatgttttataaatcgccgcctttagcagggctggttattctgctggctcttctgaatgtcgggatcacggcgctcagccgcgggaagctgagagaaaaaaaccaggacgaaatcgcaaagacttcgcagattcaaagctatcagactgagtttttatatgggatttttggtattaagactgtgggaatcgaaaaagaaacgtatcaaaaatgggatcactatctgggggaactgatcggggcataccgacgcaaggaagggtttcttaacatcgtgaactctctgacaggcacgatgcagatcatatcaccaatgctgattttatggattggtgcaatgctcgtgttcgaagggaatttatcggttggggagctggtggcatttcatgctttatccacacaattctttaatacaagcagctcgatcgtccaaaccgtcaactcggtaatcttgacgacgtcttatcttcatcggatacaggatatccttcaaagcccgattgaagagacacctggaaataaagacaactccccgattaagggggagatccggcttgaacgggtttcgttccgttacagcccgcacagcgaagaagttattaaagacgtaagcctgcatatcaagcctggcgaaaaaatcgctatcgtcggacaatccgggtccggaaaaagcacgctggcaaaacttattcttggcctctatatgcctacaaaaggcagtgtgttctatgatggcgtgaatattaaaaacaaggatttaagcgctcttcgcaaacaaattggtgttgttccccaagatgtaacgctgttcaaccgcagcattaaagacaacatttctttatacagtgaagatgttgacattgagcggatacatgaagtcgcaaaaatggcgcaaatatacgatgacattcaaaatatgccgatgggctttaatacgatgatttctgaaatggggatgaatatttcgggagggcagcgccagcggattgccttggcccgtgcactgctaaaccgccccgctgtgatgctgttggatgaagcgacgagttcgctggatcatcaaaatgaaaaaaaaatcgacgcctttttaagggagctgaaatgtacaagaattgtcatcgcgcataaactgacttcaatcatggatgccgataaaatcatcgttttggaagacggcatggttttaaatgtgggtacgcatgaaacattgctgaatgagagcaagttttacggagatttttatcgaaagttccttgaaaaagagcaatctgcagaggtgatgatgtga

SEQ ID NO: 11 - DNA, 3081 bp, Bacillus licheniformis
  gene (1)..(3081): lanM2 open reading frame of B. licheniformis
atgagcatgaaagaattcgaaatttatctgtataaagctctttacagcaatgaacgaggcggtcaaggtcaagaacatccgtccggcttttttccggaaaacggaaaaactccgtctcgtcctacggattttcacctttcttctgtccaacattcacccaatgagcctgtgcagctgcaaggcaaaatgccggaatgggctgcctgtttgtctgaaattatgaaatacaaccctaaagcggtttccgaattaaaacacccgcttccccacatgtcatttgtcaccttctttgttccttttcttttatttgcacaagaacggatgtcgaaagctttttctgaatttgagaagcaggaaggcggtctatcgggcataatcgacgctgccggctatcaagacggcatcatgtctgaacttcaccaatgccttgataagctggcgacgagaacccttatcacagagctgaatgtagcccgggaagacggccggctaaagggggcgtcaccggaagagcgatatgtttactttgttgaacaatacatttccgatcctgaaatttaccgggaatttttcgagctttaccctgtgcttggcaggctgatggctgagaaggttctcagggtgctcgagattcatgaagaaattattgggagatttttaagcgaccgcagcctgattgcgaaaaaatttaatatcgcttcccccgaattgattggatttgaaggggatttgggagattcccacaaaaacgggcagagtgtcaaagtgctggtgttaaacaacggaaagctcgtgtataaaccgcggtccttgtcaattgacgaacattacagggagctgctgaactggctgaacggacggggaatgaagtacagcctccgtgctgcggaagtgcttgacaggggaaattacggctggcaggaatttgtaaagcatgaaggctgttcttcagaagaagaactggaaagattttatttccggcagggcggacatttggcgatattgtacggattgcgctccgtcgattttcataatgaaaatatcatcgcctcaggcgaacaccccatcctgattgatctggagactctttttgacaaccatgtcagcattttcgctcaaaatcaaaacctccatgtcaccgcattggagctgaagcattccgtgctgtcttcgatgatgcttccggtcaaattcaaacatgatgaagtgctcgattttgatttaagcgggatcggcggcaaaggcggccagcagtcaaagaaagcgaagggctacgccgtcctgaattacggtgaggacaggatgtctttaaaagaaacatcgctgaccaccgaggaaaaattgaatgcgcccaaactaaatggacgtccggtgtccgccgtttcctatacggactttatcgtggaaggatttaaaaatgcttatgccattatgatgaaacataaagaagaactggcaggaccatcggggtttttgaatctgtttaagcacgacgaagtccgccacgtcttccggccgacacatgtgtacggcaagtttctcgaagcgagcacccaccctgactacttgaccgccggagataaacgagagcaattatttgattatatgtggatgctcgccaaacagtcggaaaaggcaaacgtgtttattccggacgaaattgtcgatctgctgctccatgatattccctactttactttttatgctgggggcacttccctgctcaattcaagaggggaagaatcggaaggtttttatgaaacatcaagtattgatttggcaaagaaaaaaattcaatctttctcggaaaaggatttgaatcatcagctccgctatatttctttatcaatggcgacgttgattgaaaatgtctgggaccatgcagaaagcgggcttggacagaaggaaacggtcgctgatctcggaaaagaggtcaagcatatagctgatgatttgctgcagaaggcgatctattcagagcgcggtgaaggtcctttctggatcagcaataatgccggagacgaaaaaatggtgtttttgtcgccgcttcctatggggctttacgacggaatggcagggctggcaatattttttgcacaagcaggcaaggttctgaacgagcaggtatatacggatacggcaagatcaatgatagaagaaattcaaaaggaagaaagttattgggttcaaaatgggaattcccattctgcttttttcggcacaggctcattcatttacctgtattcctatcttggcagtctatgggaagacgattccttattggaaagggcgttgaacctcattccccgagttttggaacagccgaatcaaacacaaaacccggattttatcgcaggggattcaggattgctgacagtgcttgttaatctgtacgaaatcaagcagcacccagcagtattggactctataagacaggtactgagcagattgaatgatcgaattggccgcttacttgattcaatcgagcaggatgccgtttcgttgacgggattttcacacggcttgacggggatcgcattttctatcgcaaaggcggcgaaggtgatacacgatgacagctgcaaagagcttgtcctaaagcttgtcgaagaagaggaccgctattttcaaaaggatcatctaaactggctagatttacgaaatgattcgcatacgctgtccccaagctactggtgtcatggagctcccgggattttgctggggagagcgcacattcaggcttttattcctgaattgactacccggactttaaagcttcaagaagcgcttcaaagttctttaaatctagcagactgtcaaaatcattcgctgtgccacggtttaattgggaatttgaacattctgctggatatcaaaaggctgaaccgggaacttcatgtccctgatgatatattttacatttataaaacgaaaaaccggggatggaaaacgggtttgcattccgatgtggaatcgcttggcatgtttgtcgggacggcaggaatagcctacgggcttttgcggctcctcgatgaatctgttccatccgtattaactctcgatattccgacgggcaggtga

SEQ ID NO: 12 - DNA, 219 bp, Bacillus licheniformis
  gene (1)..(219): lanA2 open reading frame of B. licheniformis
atgaaaacaatgaaaaattcagctgcccgtgaagccttcaaaggagccaatcatccggcagggatggtttccgaagaggaattgaaagctttggtaggaggaaatgacgtcaatcctgaaacaactcctgctacaacctcttcttggacttgcatcacagccggtgtaacggtttctgcttcattatgcccaacaactaagtgtacaagccgatgctag

SEQ ID NO: 13 - DNA, 225 bp, Bacillus licheniformis
  gene (1)..(225): lanA1 open reading frame of B. licheniformis
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 14 - DNA, 3159 bp, Bacillus licheniformis
  gene (1)..(3159): lanM1 open reading frame of B. licheniformis
atgaatgaaaaatccgccggatatcacgaacggcttcccgtcgcccaaactcaatccccgctcgtaaacgataagataaagtattggcgttcccttttcggcgatgatgataaatggctcaataaagcagtttcattattaagccatgaccctttgtcctccatcgcacaatcctcggtatcccagtcagtcgggctgaaagacagccgtcgcggcccatggcagaagatgcaaaagcggatctttgaaacgcccttttcctacaaggattctgctctgcaagattcagaattgctgttcgactccctgctgacccgttttgcgtctgcagcacaagatgctttggaggaacaaaatatcatactttctcctcctctttgccggcaggtgctgacacatttaaaacagacgcttcttcaaattgcccttcaaacattaatactggaactaaacattttaaggcttgaagatcaattgaagggcgacacccccgaaatgcgctatcttgatttcaatgataactttttagtcaatccaggatacctgcggaccctgttcaacgagtatcccgtattgctgcgccttctgtgcacaaaaaccgattactgggttcaaaacttttctgaactgtggaagaggctgaggcaggaccgcgaacagctgcaggctgcatttcatattgccggcgatcctgtccatattgagcttggggtgggagactcgcacaataaaggaaagatggcagccatccttacatattccgatggaaaaaagattgtctataaaccgagaagccatgatgttgacgacgcatttcaacttcttctatcatggatcaatgaccgaaattcaggcagccctttaaaaactttgagattaatcaataaaaaacggtacggatggtccgagtttattcctcacgaaacgtgccatacgaaaaaagaactggaaggctactatacacgcctcggcaaacttttggccgttttatacagcatcgatgccgttgactttcaccacgaaaacattatcgcctccggcgagcatcctgttttaatcgatcttgaatcaatttttcatcaatataaaaaacgagacgaacccggctcgaccgccgttgacaaagcaaactacattctttccagatccgtacggtctactggaatcctgccgttcaacctttacttcggaaggaaaaaccgggataaagttgtggacatcagcggaatgggggggcaggaagctcaggaatcaccgtttcaggcgcttcaaatcaaaggatttttccgcgatgacattcgcctggagcatgaccgctttgaaatcggcgaggcgaaaaatctgccgactttagatcaccagcatgtccctgtcgcagattatcttcattgtatcatcgaaggattttcagcagtataccgtctgatttctgatcatggcgaaagctacctggctacgattgaacattttaaaaactgcaccgttcgaaatattttgaagccgacagcgcactacgcctctcttttgaataaaagctaccaccctgattttctcagggatgcggtagaccgtgaagtgtttttatgccgggtggaaaagtttgaagatgcagacacagatattgcagcggcaaaaacagagctgaaagagctcattcggggagacatcccctattttctgtcgaagccttcagatacctatttgctcaatggcgaagaagaaccgattgccgcttattttgaaacgccgtccttcacaagagtaattaagaagatctcatcattttcagaccaggacttaaaggaacaagcgaatgtcatacgcatgtcgattctggctgcatataacgcgagacatgaaaaagacgcaattgatatagaccaaaatcacccgagtcctagatcaggcgccttgcagccgctcgccatcgctgagaaagcggctgacgatttggctgaaaagcgaattgaaggcaatgatggaaaggacgtcacttggatcagtacagttattgaaggcgtcgaagaaatctcttggacgatctcccctgtcagtcttgatttatataatggcaatgcaggcatcggattttttatgagctatctgagccgcttcgcaaaacggccggagacttactcgcatataaccgagcagtgtgtatttgcgattcagcgagcgttgaatgaactgaaggaaaaagaagaattcctgaagtacgccgactctggggcattcacgggggtttccggctatctgtattttctgcagcatgcgggaacggttcagaaaaaaaacgaatggatcgaactcatacatgaagctctgccagtccttgaagctgtcatcgaacaagacgaaaactgcgatatcatcagcggttctgccggtgctctaatggttctgatgtcattgtatgaacaactggatgacccggtttttctaaagctcgccgaaaagtgcgccggccatttgcttcagcataaaacaaatattgaaaacggagcggcctggaaagatcctcatacacaaaactattacacaggatttgcccacggcacttccggcatcgccgcagctttatcccgattcaataaagtgtttgattcgcaatcactgaaaaaaatcatttcgcaatgcctggcatttgaaaagcagctgtacatcgcttccgaaaaaaattggggatcaaaaggaagagaacaactgtcagttgcatggtgccatggcgctgccggcatattgttgtcgagaagcatcctccgagaaaacggagtcaatgatcccggactgcataccgacatcttgaacgctcttgaaacaactgttaagcatgggctcggcaataaccgctcattctgtcacggcgatttcggccaactcgaaatcctaagagggttcagggaagaattcagcgaactgaacaccattatacagaatacggaagatcggctgttgacatattttcaagaaaatccattcagtaaaggggtatcacgaggtgtggattcagccgggctcatgcttggtttaagcggagtcggctacggcatgctgcaatgccaatatggagaagaactgccggaactgcttcagctcagtccgcctcaagcgcttatcaaaaagaacagcaaagcttttaaaagagaaaacgtgttttaa

SEQ ID NO: 15 - DNA, 29 bp, artificial sequence
  Primer pr535
gtgctacgcgtgggaatctcccaaatccc

SEQ ID NO: 16 - DNA, 33 bp, artificial sequence
  Primer pr536
ggtgaggatccggaaaatttcgatagtttgccc

SEQ ID NO: 17 - DNA, 28 bp, artificial sequence
  Primer pr537
cttaaggatcccgcgttggcatattgat

SEQ ID NO: 18 - DNA, 28 bp, artificial sequence
  Primer pr538
cgacaccgcggaggcgataatgttttcg

SEQ ID NO: 19 - DNA, 20 bp, artificial sequence
  Primer pr539
cggaaaccgctttagggttg

SEQ ID NO: 20 - DNA, 19 bp, artificial sequence
  Primer pr540
gagcctgtgcagctgcaag

SEQ ID NO: 21 - DNA, 21 bp, artificial sequence
  Primer pr541
catactttctcctcctctttg

SEQ ID NO: 22 - DNA, 19 bp, artificial sequence
  Primer pr542
caagatagcgcatttcggg

SEQ ID NO: 23 - DNA, 20 bp, artificial sequence
  Primer pr601
gcagctccctgtaatgttcg

SEQ ID NO: 24 - DNA, 20 bp, artificial sequence
  Primer pr602
cagtagaccgtacggatctg

SEQ ID NO: 25 - DNA, 30 bp, artificial sequence
  Primer pr547
gtgctacgcgtacaacatgccaagaacagc

SEQ ID NO: 26 - DNA, 30 bp, artificial sequence
  Primer pr548
ggtgaggatccattgcagcaaaaagcggag

SEQ ID NO: 27 - DNA, 35 bp, artificial sequence
  Primer pr549
cttaaggatccaatcaaaatctatggattttcatc

SEQ ID NO: 28 - DNA, 30 bp, artificial sequence
  Primer pr550
gcacaacgcgtaaatatggccttctccgaa

SEQ ID NO: 29 - DNA, 20 bp, artificial sequence
  Primer pr551
ggatcgcatcgattgacgag

SEQ ID NO: 30 - DNA, 20 bp, artificial sequence
  Primer pr552
gctgccgatttcttcagacc

SEQ ID NO: 31 - DNA, 20 bp, artificial sequence
  Primer pr553
cagacggtaaccgtaacaac

SEQ ID NO: 32 - DNA, 20 bp, artificial sequence
  Primer pr554
gccgcaatcagctgatctcc

SEQ ID NO: 33 - DNA, 22 bp, artificial sequence
  Primer pr555
cgaactttaaagtgaactcgca

SEQ ID NO: 34 - DNA, 21 bp, artificial sequence
  Primer pr556
ctcgaattaattccgctgtcg

SEQ ID NO: 35 - DNA, 5823 bp, artificial sequence
  Plasmid pPP3932
gcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgggaattcccgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacgataagctagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacgcgt

SEQ ID NO: 36 - DNA, 7914 bp, artificial sequence
  Plasmid pBKQ1697
cgcgtgggaatctcccaaatccccttcaaatccaatcaattcgggggaagcgatattaaattttttcgcaatcaggctgcggtcgcttaaaaatctcccaataatttcttcatgaatctcgagcaccctgagaaccttctcagccatcagcctgccaagcacagggtaaagctcgaaaaattcccggtaaatttcaggatcggaaatgtattgttcaacaaagtaaacatatcgctcttccggtgacgccccctttagccggccgtcttcccgggctacattcagctctgtgataagggttctcgtcgccagcttatcaaggcattggtgaagttcagacatgatgccgtcttgatagccggcagcgtcgattatgcccgatagaccgccttcctgcttctcaaattcagaaaaagctttcgacatccgttcttgtgcaaataaaagaaaaggaacaaagaaggtgacaaatgacatgtggggaagcgggtgttttaattcggaaaccgctttagggttgtatttcataatttcagacaaacaggcagcccattccggcattttgccttgcagctgcacaggctcattgggtgaatgttggacagaagaaaggtgaaaatccgtaggacgagacggagtttttccgttttccggaaaaaagccggacggatgttcttgaccttgaccgcctcgttcattgctgtaaagagctttatacagataaatttcgaattctttcatgctcattctcgtcatccctttggcgaagaaaaccatccctgcaactccgttgcaaggatggattcttttgaattttttatgattccctagcatcggcttgtacacttagttgttgggcataatgaagcagaaaccgttacaccggctgtgatgcaagtccaagaagaggttgtagcaggagttgtttcaggattgacgtcatttcctcctaccaaagctttcaattcctcttcggaaaccatccctgccggatgattggctcctttgaaggcttcacgggcagctgaatttttcattgttttcatgattctcacctcctagaacgggcaaactatcgaaattttccggatcccgcgttggcatattgatagaaaaagaggtcgatagaatgaatgaaaaatccgccggatatcacgaacggcttcccgtcgcccaaactcaatccccgctcgtaaacgataagataaagtattggcgttcccttttcggcgatgatgataaatggctcaataaagcagtttcattattaagccatgaccctttgtcctccatcgcacaatcctcggtatcccagtcagtcgggctgaaagacagccgtcgcggcccatggcagaagatgcaaaagcggatctttgaaacgcccttttcctacaaggattctgctctgcaagattcagaattgctgttcgactccctgctgacccgttttgcgtctgcagcacaagatgctttggaggaacaaaatatcatactttctcctcctctttgccggcaggtgctgacacatttaaaacagacgcttcttcaaattgcccttcaaacattaatactggaactaaacattttaaggcttgaagatcaattgaagggcgacacccccgaaatgcgctatcttgatttcaatgataactttttagtcaatccaggatacctgcggaccctgttcaacgagtatcccgtattgctgcgccttctgtgcacaaaaaccgattactgggttcaaaacttttctgaactgtggaagaggctgaggcaggaccgcgaacagctgcaggctgcatttcatattgccggcgatcctgtccatattgagcttggggtgggagactcgcacaataaaggaaagatggcagccatccttacatattccgatggaaaaaagattgtctataaaccgagaagccatgatgttgacgacgcatttcaacttcttctatcatggatcaatgaccgaaattcaggcagccctttaaaaactttgagattaatcaataaaaaacggtacggatggtccgagtttattcctcacgaaacgtgccatacgaaaaaagaactggaaggctactatacacgcctcggcaaacttttggccgttttatacagcatcgatgccgttgactttcaccacgaaaacattatcgcctccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgggaattcccgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacgataagctagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttca

SEQ ID NO: 37 - DNA, 7892 bp, artificial sequence
  Plasmid pBKQ1699
gcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgggaattcccgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacgataagctagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacgcgtaaatatggccttctccgaaacggaatgacggcacacgcgaattcagattctcaaaccagttatgatggaatgtaatcgtcctgttgtaattatcgctgtccgatgaacccatcagcattgatttccatccatcgtgcacatagttccaagagaatgtaatatattccgcatctcttttgacgtcaaataatccgtcatagtaatctttgtcaacgttcaggctgtggtaaagctcattatgatcaacccaaatgtttttagaagggccttcaatgccgatcgcgtctttatcgcctgaggcgacctcgtgaattttcaagttgcggatgatgatgttgttggcccgccatattttgatgccgatccctttgagttcccctttggtccctgatccgacaatcgatacgtttgacacgtctttgacgtcaatctttgatgcggatgtatttgatgttgtaatggtgccgttgacataaatttttaaaggcgtatttgcattcttattttttaatgccgcaatcagctgatctcccgttgttacggttaccgtctgaccgccttctccgcccgttgttccgccgtttagtgcggcaaagccttttaagctgaagtcggcaagcggatttactttgcccgagtttaaggcagaagctgcttctgccgaaaccgccgctgtcaatgacccgacaacccctaatacaaagataaagatgatgctgattaatttcttcattatattcctcctattgtttcttttttgatgccaacgctgaatgggaagcgcttacaattacggcggatactgaatggtgggcaccgatcatccctcccttttattggaatcagagagagcatagcatatatttctgaattgtctgaataatcatgttgtgagttggatttgctcataagctggtgattatgccaaatgacacgaatctgagaataagcgcatttggatgaaggtctttcatcgtaatgttgcaaaaaaaatctatggatgaaaatccatagattttgattggatccattgcagcaaaaagcggagctcgatttacagctccgctttctcctgttctgtgctgttgtatgttcgctccacgactgaagcaagcgtatcgatgaaccgcggatcagtgttcggcataggcggacggtgatagcttgcgccaagctcgtccgtaaccactttgcactcatagtcgttgtcatacagaacttccaggtggtcagatacaaaaccgactggtgcgtaaatgaatgaccggaagccttcttcatacagttcctttgttaagtcttggacgtccgggccgagccaaggatcgggcgtgttgccctcgctttgccagcctacggcgatctggtcaaatgacagccgttctccgattagttgtgctgttttctcgagctgatccgggtaaggatcgtcatgctccctgattttttccggcaggctgtgtgctgagaatatgacggccgctttttcccgctctttttcagacatgtcgtttaaaatgctgccgatttcttcagaccaatagcgaataaagccttcttcctgataccactcgtcaatcgatgcgatccgtggtccgccgattgctgctgcggcctgtttggcgcgccgattgtatacttcggtgctgaaggtggaatagtggggagctaagacgacggaaacggcctcttcaatttggtcttttttcatttgttcaaccgcatcctcgataaacggagagatgtgttttaagccgagatacatcacgtattcgactttatcttggcgctcgttcaacgttttttccagctcttttgcctgagcgagcgtaattttcgcgagcggagagattccgccgatatgcttgtagcgttttttcaagtcttcaatcatatcatcggacggtcgttttccatgacggatatgcgtgtaatacggaatgatatcttcttcctgataaggggttccgtatgccatgactaacaggccgactttttttcgttccatatgtcttcacctctgctgctgttcttggcatgttgtgctgttcttggcatgttgtacgcgt

SEQ ID NO: 38 - DNA, 9067 bp, artificial sequence
  Plasmid pBKQ1751
acgcgtacaacatgccaagaacagcacaacatgccaagaacagcagcagaggtgaagacatatggaacgaaaaaaagtcggcctgttagtcatggcatacggaaccccttatcaggaagaagatatcattccgtattacacgcatatccgtcatggaaaacgaccgtccgatgatatgattgaagacttgaaaaaacgctacaagcatatcggcggaatctctccgctcgcgaaaattacgctcgctcaggcaaaagagctggaaaaaacgttgaacgagcgccaagataaagtcgaatacgtgatgtatctcggcttaaaacacatctctccgtttatcgaggatgcggttgaacaaatgaaaaaagaccaaattgaagaggccgtttccgtcgtcttagctccccactattccaccttcagcaccgaagtatacaatcggcgcgccaaacaggccgcagcagcaatcggcggaccacggatcgcatcgattgacgagtggtatcaggaagaaggctttattcgctattggtctgaagaaatcggcagcattttaaacgacatgtctgaaaaagagcgggaaaaagcggccgtcatattctcagcacacagcctgccggaaaaaatcagggagcatgacgatccttacccggatcagctcgagaaaacagcacaactaatcggagaacggctgtcatttgaccagatcgccgtaggctggcaaagcgagggcaacacgcccgatccttggctcggcccggacgtccaagacttaacaaaggaactgtatgaagaaggcttccggtcattcatttacgcaccagtcggttttgtatctgaccacctggaagttctgtatgacaacgactatgagtgcaaagtggttacggacgagcttggcgcaagctatcaccgtccgcctatgccgaacactgatccgcggttcatcgatacgcttgcttcagtcgtggagcgaacatacaacagcacagaacaggagaaagcggagctgtaaatcgagctccgctttttgctgcaatggatccttctatcttttatagggtcattagagtatacttatttgtcctataaactatttagcagcataatagatttattgaataggtcattaagttgagcgtattagaggaggaaaatcttggggaaatatttgaagaaccgtggatatttttaaaatatatacttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgaaattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttctacagtcggttttctaatgtcactaacctgccccgttagctgaagaaggtttttatattacagctccgtgagagagaagcgaacacttgattttttaattttctatcttttataggtcattagagtatacttatttgtcctataaactatttagcagcataatagatttattgaataggtcatttaagttgagcatattagaggaggaaaatcagatccgaaccattgatccaatcaaaatctatggattttcatccatagattttttttgcaacattacgatgaaagaccttcatccaaatgcgcttattctcagattcgtgtcatttggcataatcaccagcttatgagcaaatccaactcacaacatgattattcagacaattcagaaatatatgctatgctctctctgattccaataaaagggagggatgatcggtgcccaccattcagtatccgccgtaattgtaagcgcttcccattcagcgttggcatcaaaaaagaaacaataggaggaatataatgaagaaattaatcagcatcatctttatctttgtattaggggttgtcgggtcattgacagcggcggtttcggcagaagcagcttctgccttaaactcgggcaaagtaaatccgcttgccgacttcagcttaaaaggctttgccgcactaaacggcggaacaacgggcggagaaggcggtcagacggtaaccgtaacaacgggagatcagctgattgcggcattaaaaaataagaatgcaaatacgcctttaaaaatttatgtcaacggcaccattacaacatcaaatacatccgcatcaaagattgacgtcaaagacgtgtcaaacgtatcgattgtcggatcagggaccaaaggggaactcaaagggatcggcatcaaaatatggcgggccaacaacatcatcatccgcaacttgaaaattcacgaggtcgcctcaggcgataaagacgcgatcggcattgaaggcccttctaaaaacatttgggttgatcataatgagctttaccacagcctgaacgttgacaaagattactatgacggattatttgacgtcaaaagagatgcggaatatattacattctcttggaactatgtgcacgatggatggaaatcaatgctgatgggttcatcggacagcgataattacaacaggacgattacattccatcataactggtttgagaatctgaattcgcgtgtgccgtcattccgtttcggagaaggccatatttacgcgtgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgtcgaattgcatctgaccgaattttacgtttccctgaataattctcatcaatcgtttcatcaattttatctttatactttatattttgtgcgttaatcaaatcataatttttatatgtttcctcatgatttatgtctttattattatagtttttattctctctttgattatgtctttgtatcccgtttgtattacttgatcctttaactctggcaaccctcaaaattgaatgagacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgatttacatatgagttatgcagtttgtagaatgcaaaaagtgaaatcagctggactaaaaggcagagctcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgggaattcccgattatgtcttttgcgcagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcatcaaatttaaacttcattgcataatccattaaactcttaaattctacgattccttgttcatcaataaactcaatcatttctttaattaatttatatctatctgttgttgttttctttaataattcatcaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacaatggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgctgcgtccattaaaaatcctagagctttgcaaccgaaagttaatagctgtcgctactactttcgcttacgctctaagtatattttaaggactgtcacacgcaaaaagttttctcggcataaaagtacctctacatctctaaatcgtctgtacgctgtttctcacgctttctatcgatgaccttcatgttaacccctcaagctcaggggagtaaacaggagacaagtgcttagttatttcgtcaccaaatgatgttattccgcgaaatataatgaccctcttgataacccaagagggcattttttacgataaagaagatttagcttcaaataaaacctatctattttatttatctttcaagctcaataaaaagccgcggtaaatagcaataaattggccttttttatcggcaagctcttttaggtttttcgcatgtattgcgatatgc

SEQ ID NO: 39 - protein, 382 aa, artificial sequence
  AprH expression construct.
  SIGNAL (1)..(29): AprL signal peptide from B. licheniformis
  PROPEP (30)..(113): AprH pro-peptide from Bacillus clausii
  mat_peptide (114)..(382): AprH mature peptide from Bacillus clausii
MMRKKSFWLGMLTAFMLVFTMAFSDSASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 40 - DNA, 2182 bp, artificial sequence
  PCR product “lig-PCR lanA1 flanks”
gtgctacgcgtgggaatctcccaaatccccttcaaatccaatcaattcgggggaagcgatattaaattttttcgcaatcaggctgcggtcgcttaaaaatctcccaataatttcttcatgaatctcgagcaccctgagaaccttctcagccatcagcctgccaagcacagggtaaagctcgaaaaattcccggtaaatttcaggatcggaaatgtattgttcaacaaagtaaacatatcgctcttccggtgacgccccctttagccggccgtcttcccgggctacattcagctctgtgataagggttctcgtcgccagcttatcaaggcattggtgaagttcagacatgatgccgtcttgatagccggcagcgtcgattatgcccgatagaccgccttcctgcttctcaaattcagaaaaagctttcgacatccgttcttgtgcaaataaaagaaaaggaacaaagaaggtgacaaatgacatgtggggaagcgggtgttttaattcggaaaccgctttagggttgtatttcataatttcagacaaacaggcagcccattccggcattttgccttgcagctgcacaggctcattgggtgaatgttggacagaagaaaggtgaaaatccgtaggacgagacggagtttttccgttttccggaaaaaagccggacggatgttcttgaccttgaccgcctcgttcattgctgtaaagagctttatacagataaatttcgaattctttcatgctcattctcgtcatccctttggcgaagaaaaccatccctgcaactccgttgcaaggatggattcttttgaattttttatgattccctagcatcggcttgtacacttagttgttgggcataatgaagcagaaaccgttacaccggctgtgatgcaagtccaagaagaggttgtagcaggagttgtttcaggattgacgtcatttcctcctaccaaagctttcaattcctcttcggaaaccatccctgccggatgattggctcctttgaaggcttcacgggcagctgaatttttcattgttttcatgattctcacctcctagaacgggcaaactatcgaaattttccggatcccgcgttggcatattgatagaaaaagaggtcgatagaatgaatgaaaaatccgccggatatcacgaacggcttcccgtcgcccaaactcaatccccgctcgtaaacgataagataaagtattggcgttcccttttcggcgatgatgataaatggctcaataaagcagtttcattattaagccatgaccctttgtcctccatcgcacaatcctcggtatcccagtcagtcgggctgaaagacagccgtcgcggcccatggcagaagatgcaaaagcggatctttgaaacgcccttttcctacaaggattctgctctgcaagattcagaattgctgttcgactccctgctgacccgttttgcgtctgcagcacaagatgctttggaggaacaaaatatcatactttctcctcctctttgccggcaggtgctgacacatttaaaacagacgcttcttcaaattgcccttcaaacattaatactggaactaaacattttaaggcttgaagatcaattgaagggcgacacccccgaaatgcgctatcttgatttcaatgataactttttagtcaatccaggatacctgcggaccctgttcaacgagtatcccgtattgctgcgccttctgtgcacaaaaaccgattactgggttcaaaacttttctgaactgtggaagaggctgaggcaggaccgcgaacagctgcaggctgcatttcatattgccggcgatcctgtccatattgagcttggggtgggagactcgcacaataaaggaaagatggcagccatccttacatattccgatggaaaaaagattgtctataaaccgagaagccatgatgttgacgacgcatttcaacttcttctatcatggatcaatgaccgaaattcaggcagccctttaaaaactttgagattaatcaataaaaaacggtacggatggtccgagtttattcctcacgaaacgtgccatacgaaaaaagaactggaaggctactatacacgcctcggcaaacttttggccgttttatacagcatcgatgccgttgactttcaccacgaaaacattatcgcctccgcggtgtcg

SEQ ID NO: 41 - DNA, 15196 bp, artificial sequence
  The lan gene cluster.
gttatgacgtgacaatatcccgcttttgaaaaacccaaaaggagacagccagaaaaagaataaaataaacagcaatcacagccgctgaaaatgaaagagtcatatcttgaataaacggctccccatcaatgtattgactgagatctgaatttgcaaagatcgtatacttggtccaatcaaattttgccgctaaaaattgagtaatcgcagaccctgtaaaaaagacgaacagcgaaagaccgacagagacgaccgcgcttcttaggatgaccgaaatcataaaggccagcgtggccagcacaataatattcaataaagcggctccatagtctaaggctaaaaacttcagcattgacatctttatcacttcgccgcttcgataggacaaatagtattggcttccctccaaacctagcagggcataccctaaaacaaacgcaaataaaagtgtcgcagcaaacataaatacagtgttgagcaagatcgacaagtatttccccaatagataatgccaacgcttgaccggtgttgcaatcgcaaatttaatagttcctttgctgtgctcaagcgcgatggagttcgctgccagcacgattgcaatcaacgcgattaatgttgtgatcggttttaactccttcagcatcgtccaaacattgtatttcgcagaaggcggcaaattatgctcaagccggtattgattgaccgcgatctcttcttttaaaaattggtatttgggaaccgctgggctcagctctctcatttctctttcatactgagcgttttgaacggttaattgctctttccagttacctgtctccttctccttttcgccattgctgaccatccatgcaacagcgattgtcatgacaagcagcaagcttaacacaatccaggtacttttcgtcaccatcagcctgtaaagttcatttttcaagaccctcatgccggtcaccccttattgatcaattccagaaagctttgttctatcgactgtttttcctgcttcatatgcagaactcttattttgttctccattaacagattaacgactttcataatgtcttcttccatgcaaaggacataaatctcatttttatcttccgtgagaccgcctgcatactggtactcattcaacaggtcagccgcttcatcggcgtggccgtttaatgtaagccgataacgaaccgtatcggtatcgcggtgattgctcttaatttgcaggagttctccgtttttcatgacgccgtattcatcacaaatcatctcaacctcagacagaagatgactcgaaatcaaaatcgttttgttgaaggttttcgccaagtactgaaggtgttcgcgcaaatcaatgattccctgaggatcaaggccgtttgtcggttcatcgagaatgagaaattccggatcatgcaagagcgcgacggcaaggcccaaacgctgtttcatgcctaatgaatacgttttaactggtttatgaatacttcctgtcaaatcaacaagctgaacgatctcttctatgcgctcctttgacacgtggctgtggaatccgccgagatatttaaggttttggtatcctgttaagtgctcataaaaggatgggttttcaacaacagaaccgactcgggctgccgcttcctcgtaattgcttttaactgaatagccgtcgatgcggatatctcctgatgtcggctttgccatcccgacaatcattttgataagtgttgtcttgccagcgccgtttggcccgagcagcccaaaaattgtacctggggcaatttgaaatgtgatattttggacaattggtctgccttttattcgtttacttacaccttcaaattcgacttgactcattttgtattcctcctttttggtatggcctgatctgtttcaatagaagagaaaaaaagagcagcgaaaccgccgtgaaccaaacaatcgaaccgataagaacggcaactgacttctccattgcaatgagggccgtcacaggaaaagaccatggaaagagaagccctaaaggggattgcgaaacgccgaaactcagcagtgaacaaatgaatacgataatgtttaatgaggtcttttccttactggtgaatttaagccataggaccgtacagatcagcggaaaggttgacaagagattgagaatgaagcccagcatccaagaaaaccaaggggcaggcccggggaaccccgatattaaaccgaaggcaagcgattcgatcacacagacggcatataatgcggctgcgaacagtccaataaacaaaaccttagcaaaaagcatggctgcgaaaggctgcggcgataagattcgattcagccacactcggtttttcagttcatccttatgcaaaaatactgtgaccgaagagacaagccagggataaatcgtgctgaataccaacagcaatgtaagatgccttaacgatgtccattcattccctcccgcaaacggggtgattcgttttgccataaaccctgtaaacactaaagatgcggtaagaatgaatatgctcgtcattgtaaaatcagagttcctgagtttgatccattccgctttgagcaaatttttcacggatcttttcctccccgctccttaagatccaacacatacagaccgctatataacccgccgaaataagataaactgccaggcctatatcgttcactgaatattcagtcaacaaaagccaagaaactccccacggcaaataaagtgttccactttgcataaaaaacggcgtcagcgtcatcaacagcacaaatagatattctgcctttaacgtcttcagaaaaaactggacggaagacatgatcaccgccagtatcgcaacagcggctaaacgctctggcgaaaacaaaacatccttttgaaacagcagactgactgccaaacagccgtaaagcaagaccagcgctgagacgatagttatcacggcgattccagtcagcacccaagtgaacaggcgaacgggataccgcaagataaaagcttgatgcatcatgcgtccaaataatcttctgcctgtaatataagtcagccaaaccgagacgacataaacagtcattccctccatgctacggatttctttatttgccccgtttttataaaagagaagataattaagcaaaacggcgagcactgtgatgatcataaccgtacatgcaaacgtctgaaatttcattctctttttcaccgcttcgggacagcttaaccaaaaataaactttaaacaatttcatttaaacgcccgctttctatggctagaaaacgatcaaacaattcgataaaagtttcgttccaatggcttgatgccatgatggtcccgtcataccgccttatgatgtctacaacgctttcctgcgcttttcggtcgagcccatttaaaggctcatccaatattaagatggaaggctgatgaaataaggccattgcgatcttcagcttttgcctcatgcctgttgaatagtactgtactttcgtgtcatcagccggattgataccgaaccgctctagttcagaatagatccggtcatcgtcatctattccgtgaagcaaggcatggactctcagattgtccgttccagtaagatgctcatgaagcggcacttcgtcactgacatagccgatatgcccgacagcagcttccctgttttggataatcgattgaccgtaaatcaaaatttcgcccgcaaataaagacaagccggtaattgctttaagaaacgtcgattttcccgatccgttttctcctttgagcaaaacccggctccctttgtccacagcaaggctgacgttttcaagaagtttcctgtctcccgcccgcacctcaacatgtttcatgacaattggaagctcaaccatggctacacccttgaacgaattcccaacaaaatgaagaatccaatgaaatacggcagcgaaaaagccagtgtgaccatgtttttataattggcgaacgacaaatcgacccatgaacttgtaaaaataacggcggatattccaagtgtaagcaaaaagaacatcacccgaaagaatttgttttgaatatatgccccgcgttcatcctttccgtcaaggctgattaaaataatgattccgataaaccccgcaaatcctgcgacagcaccaattgaattgatcatttctagcatcagcgatcatcctcctctagttggtattgaaatacctcgttgatgtccttctcaaataacgcggcaatgcgaaaccccattaacagggatggtttgtatttattcttttcaatggaaatcacagtttgtcttgttacacccaatagatcggcaagctgcttttgcgtccaccctttctctgctcgcagcacaggaatccggttggttaacaccccgctttgcttttcaatctcaatcaccctctttatttgcaaatcatttacatatatagagtacaatgttttttacacattgtaaagagtttataacaaaaagtaaaaagttttttaccaaaacagactaaaaaaagatcttcacgtgatgattgtgtgaagatcttttcgacaatcaagatttaggtttttcaaaaatcagttcgatagaagaaacctggcctccggcccttgatggaacagaaaagacgctgtgaagcttccagcctttttcagcatattcatgaatcacatcagaataatttttctcaagttttccgttgaatgctcctacagcgattgaaactgtttgatattcatacatacccttcactccttgttcatcattttcagcgcttgataagcgcgtacttccccgtagccgtatataacatctttccccggtttgcccaggtctaatgccgatttcctcaggatgtgatgcacttgtttagccgatggcttcctgtgatggcgctcccggtattcagatataaccagtgcagctgttccagctacctgcggggcggccaagcttgttccgtatgagagggaatatccctttgggatgtttagcatctggtctaatgcggtgttgtccttccctttcggatacgttgtcagtacgaggtctgtcacacgaaccatcccatcctgatcatacgtttctcccaaatatcctcccggggctgtgaattcaatttcccgcccttgattggagtatggggaaatattgttgcttttcatattgctgctgactgatacgacgtgcttcaaagcgctcggcaaatgcaccttgttttccgttttcctcatctcatcaaggttgactcctttattgccggcggaagagaagaccagcacctgatgctttgcagcgtagtttgcagccctttgataagctttcatcagcacttttccttccctgtcaagagaagtatagcttcccgtactgatgttgatgacttcatgcccgtcattgactgcatcaaccatcgccttcatgatgttgtagctgtctccgcctttttcgtccagcacttgataaggcgttatcgtcacatctggcgcaatgatattgatcatcccggctgtttgtgttccgtgtcctgtcggatctccggatacaggcttgccgtcaatgtaattccagccgccatttgtattgacggacgcttttaagtccggatggtcaagatccagtccgctgtcaatcaaggcaactttgacgtccggatgccctctttcgatttgatacgtgcgataggaatccgtctgctttgcagcgaaccatagatattcttttagttccaaaataccgtcaagcccccatgcaggcgaaccgctgatgactgattccgtttcgtttactgctgtatttgcaatcggcttttcaatgacgtcagccccgactttttcagcaagcttctttgtaaaaggagcaagcttttgtttttcaccggtcacttttgcaagtccgatttcttcaatgacatcggcatggatgtgaaattgttccgccgtgtccagcagtttcgatttatcttttacatgttcaagcaaaagatactgttctcctgcttgttcttttgcttgagccgtctttccgccgaccggcagcaggactgcaaaacataagagaaaaatatatattcttttcacatcatcacctctgcagattgctctttttcaaggaactttcgataaaaatctccgtaaaacttgctctcattcagcaatgtttcatgcgtacccacatttaaaaccatgccgtcttccaaaacgatgattttatcggcatccatgattgaagtcagtttatgcgcgatgacaattcttgtacatttcagctcccttaaaaaggcgtcgatttttttttcattttgatgatccagcgaactcgtcgcttcatccaacagcatcacagcggggcggtttagcagtgcacgggccaaggcaatccgctggcgctgccctcccgaaatattcatccccatttcagaaatcatcgtattaaagcccatcggcatattttgaatgtcatcgtatatttgcgccatttttgcgacttcatgtatccgctcaatgtcaacatcttcactgtataaagaaatgttgtctttaatgctgcggttgaacagcgttacatcttggggaacaacaccaatttgtttgcgaagagcgcttaaatccttgtttttaatattcacgccatcatagaacacactgccttttgtaggcatatagaggccaagaataagttttgccagcgtgctttttccggacccggattgtccgacgatagcgattttttcgccaggcttgatatgcaggcttacgtctttaataacttcttcgctgtgcgggctgtaacggaacgaaacccgttcaagccggatctcccccttaatcggggagttgtctttatttccaggtgtctcttcaatcgggctttgaaggatatcctgtatccgatgaagataagacgtcgtcaagattaccgagttgacggtttggacgatcgagctgcttgtattaaagaattgtgtggataaagcatgaaatgccaccagctccccaaccgataaattcccttcgaacacgagcattgcaccaatccataaaatcagcattggtgatatgatctgcatcgtgcctgtcagagagttcacgatgttaagaaacccttccttgcgtcggtatgccccgatcagttcccccagatagtgatcccatttttgatacgtttctttttcgattcccacagtcttaataccaaaaatcccatataaaaactcagtctgatagctttgaatctgcgaagtctttgcgatttcgtcctggtttttttctctcagcttcccgcggctgagcgccgtgatcccgacattcagaagagccagcagaataaccagccctgctaaaggcggcgatttataaaacatataaaacgagataaataaaagtgcgccaaaatcgagtacgccgaggaccaattgggaagacatcatatccctgacaatccggaggcttgtcgcgcggaaaaggagatccccgaacgacctcaattgaaagaattgatagggaagcttcagaatatgcgagaaaaatgttctcatcattcgtttgtcaaggaagttatttaatttaatgataaattgacccctgatcaattgaaccgtgccttgaacgagcattaaaacaagaacgcccgtcataaatgtttgcagcagagcctgctgattttgagcaaggatgtcatcgatcaggtattgcacgagcatcggtatgcctaatgaaacgagctgaaagatcatcgcaaacaacagcacggtcgcaagcagagaaggggattcttttaaatgaaaaagaaaatgcctccagacgggcggttttttcttcggcctgatccgctcggtagggatcatctccagcacgatcccgctgtaattttcgagaaaagcttcgatactgagtttttttcgtccaatcgcaggatcgatgatgttgacatgccggtcttttatttgatcaataaccacataatgattatcggaccaaaaagcgattgccggcagacgcacatgtggaagttctgccgcgcctgccctgtacacctttgcgtcaaatcccagattttcgctgagctgacgcatatgaaaaagggtcataccgtccctgccgcatcccgataaatccctcagctcatgcagagtatgatgcgaaccataccgtccggctaccatcgccatacagcacagtccgcattccgtctgctgcatctgttctataaacggtgtcttatgaaaaaacaagccttatctcacctgcccgtcggaatatcgagagttaatacggatggaacagattcatcgaggagccgcaaaagcccgtaggctattcctgccgtcccgacaaacatgccaagcgattccacatcggaatgcaaacccgttttccatccccggtttttcgttttataaatgtaaaatatatcatcagggacatgaagttcccggttcagccttttgatatccagcagaatgttcaaattcccaattaaaccgtggcacagcgaatgattttgacagtctgctagatttaaagaactttgaagcgcttcttgaagctttaaagtccgggtagtcaattcaggaataaaagcctgaatgtgcgctctccccagcaaaatcccgggagctccatgacaccagtagcttggggacagcgtatgcgaatcatttcgtaaatctagccagtttagatgatccttttgaaaatagcggtcctcttcttcgacaagctttaggacaagctctttgcagctgtcatcgtgtatcaccttcgccgcctttgcgatagaaaatgcgatccccgtcaagccgtgtgaaaatcccgtcaacgaaacggcatcctgctcgattgaatcaagtaagcggccaattcgatcattcaatctgctcagtacctgtcttatagagtccaatactgctgggtgctgcttgatttcgtacagattaacaagcactgtcagcaatcctgaatcccctgcgataaaatccgggttttgtgtttgattcggctgttccaaaactcggggaatgaggttcaacgccctttccaataaggaatcgtcttcccatagactgccaagataggaatacaggtaaatgaatgagcctgtgccgaaaaaagcagaatgggaattcccattttgaacccaataactttcttccttttgaatttcttctatcattgatcttgccgtatccgtatatacctgctcgttcagaaccttgcctgcttgtgcaaaaaatattgccagccctgccattccgtcgtaaagccccataggaagcggcgacaaaaacaccattttttcgtctccggcattattgctgatccagaaaggaccttcaccgcgctctgaatagatcgccttctgcagcaaatcatcagctatatgcttgacctcttttccgagatcagcgaccgtttccttctgtccaagcccgctttctgcatggtcccagacattttcaatcaacgtcgccattgataaagaaatatagcggagctgatgattcaaatccttttccgagaaagattgaatttttttctttgccaaatcaatacttgatgtttcataaaaaccttccgattcttcccctcttgaattgagcagggaagtgcccccagcataaaaagtaaagtagggaatatcatggagcagcagatcgacaatttcgtccggaataaacacgtttgccttttccgactgtttggcgagcatccacatataatcaaataattgctctcgtttatctccggcggtcaagtagtcagggtgggtgctcgcttcgagaaacttgccgtacacatgtgtcggccggaagacgtggcggacttcgtcgtgcttaaacagattcaaaaaccccgatggtcctgccagttcttctttatgtttcatcataatggcataagcatttttaaatccttccacgataaagtccgtataggaaacggcggacaccggacgtccatttagtttgggcgcattcaatttttcctcggtggtcagcgatgtttcttttaaagacatcctgtcctcaccgtaattcaggacggcgtagcccttcgctttctttgactgctggccgcctttgccgccgatcccgcttaaatcaaaatcgagcacttcatcatgtttgaatttgaccggaagcatcatcgaagacagcacggaatgcttcagctccaatgcggtgacatggaggttttgattttgagcgaaaatgctgacatggttgtcaaaaagagtctccagatcaatcaggatggggtgttcgcctgaggcgatgatattttcattatgaaaatcgacggagcgcaatccgtacaatatcgccaaatgtccgccctgccggaaataaaatctttccagttcttcttctgaagaacagccttcatgctttacaaattcctgccagccgtaatttcccctgtcaagcacttccgcagcacggaggctgtacttcattccccgtccgttcagccagttcagcagctccctgtaatgttcgtcaattgacaaggaccgcggtttatacacgagctttccgttgtttaacaccagcactttgacactctgcccgtttttgtgggaatctcccaaatccccttcaaatccaatcaattcgggggaagcgatattaaattttttcgcaatcaggctgcggtcgcttaaaaatctcccaataatttcttcatgaatctcgagcaccctgagaaccttctcagccatcagcctgccaagcacagggtaaagctcgaaaaattcccggtaaatttcaggatcggaaatgtattgttcaacaaagtaaacatatcgctcttccggtgacgccccctttagccggccgtcttcccgggctacattcagctctgtgataagggttctcgtcgccagcttatcaaggcattggtgaagttcagacatgatgccgtcttgatagccggcagcgtcgattatgcccgatagaccgccttcctgcttctcaaattcagaaaaagctttcgacatccgttcttgtgcaaataaaagaaaaggaacaaagaaggtgacaaatgacatgtggggaagcgggtgttttaattcggaaaccgctttagggttgtatttcataatttcagacaaacaggcagcccattccggcattttgccttgcagctgcacaggctcattgggtgaatgttggacagaagaaaggtgaaaatccgtaggacgagacggagtttttccgttttccggaaaaaagccggacggatgttcttgaccttgaccgcctcgttcattgctgtaaagagctttatacagataaatttcgaattctttcatgctcattctcgtcatccctttggcgaagaaaaccatccctgcaactccgttgcaaggatggattcttttgaattttttatgattccctagcatcggcttgtacacttagttgttgggcataatgaagcagaaaccgttacaccggctgtgatgcaagtccaagaagaggttgtagcaggagttgtttcaggattgacgtcatttcctcctaccaaagctttcaattcctcttcggaaaccatccctgccggatgattggctcctttgaaggcttcacgggcagctgaatttttcattgttttcatgattctcacctcctagaacgggcaaactatcgaaattttcctataatttagaattgactcgccgttccagtcaaattatactataagtacatcaagaaatcgacaaaaaattataaattttctaggaggtggaatatatgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaagttcccaacgcgggggccctgctcccgcgttggcatattgatagaaaaagaggtcgatagaatgaatgaaaaatccgccggatatcacgaacggcttcccgtcgcccaaactcaatccccgctcgtaaacgataagataaagtattggcgttcccttttcggcgatgatgataaatggctcaataaagcagtttcattattaagccatgaccctttgtcctccatcgcacaatcctcggtatcccagtcagtcgggctgaaagacagccgtcgcggcccatggcagaagatgcaaaagcggatctttgaaacgcccttttcctacaaggattctgctctgcaagattcagaattgctgttcgactccctgctgacccgttttgcgtctgcagcacaagatgctttggaggaacaaaatatcatactttctcctcctctttgccggcaggtgctgacacatttaaaacagacgcttcttcaaattgcccttcaaacattaatactggaactaaacattttaaggcttgaagatcaattgaagggcgacacccccgaaatgcgctatcttgatttcaatgataactttttagtcaatccaggatacctgcggaccctgttcaacgagtatcccgtattgctgcgccttctgtgcacaaaaaccgattactgggttcaaaacttttctgaactgtggaagaggctgaggcaggaccgcgaacagctgcaggctgcatttcatattgccggcgatcctgtccatattgagcttggggtgggagactcgcacaataaaggaaagatggcagccatccttacatattccgatggaaaaaagattgtctataaaccgagaagccatgatgttgacgacgcatttcaacttcttctatcatggatcaatgaccgaaattcaggcagccctttaaaaactttgagattaatcaataaaaaacggtacggatggtccgagtttattcctcacgaaacgtgccatacgaaaaaagaactggaaggctactatacacgcctcggcaaacttttggccgttttatacagcatcgatgccgttgactttcaccacgaaaacattatcgcctccggcgagcatcctgttttaatcgatcttgaatcaatttttcatcaatataaaaaacgagacgaacccggctcgaccgccgttgacaaagcaaactacattctttccagatccgtacggtctactggaatcctgccgttcaacctttacttcggaaggaaaaaccgggataaagttgtggacatcagcggaatgggggggcaggaagctcaggaatcaccgtttcaggcgcttcaaatcaaaggatttttccgcgatgacattcgcctggagcatgaccgctttgaaatcggcgaggcgaaaaatctgccgactttagatcaccagcatgtccctgtcgcagattatcttcattgtatcatcgaaggattttcagcagtataccgtctgatttctgatcatggcgaaagctacctggctacgattgaacattttaaaaactgcaccgttcgaaatattttgaagccgacagcgcactacgcctctcttttgaataaaagctaccaccctgattttctcagggatgcggtagaccgtgaagtgtttttatgccgggtggaaaagtttgaagatgcagacacagatattgcagcggcaaaaacagagctgaaagagctcattcggggagacatcccctattttctgtcgaagccttcagatacctatttgctcaatggcgaagaagaaccgattgccgcttattttgaaacgccgtccttcacaagagtaattaagaagatctcatcattttcagaccaggacttaaaggaacaagcgaatgtcatacgcatgtcgattctggctgcatataacgcgagacatgaaaaagacgcaattgatatagaccaaaatcacccgagtcctagatcaggcgccttgcagccgctcgccatcgctgagaaagcggctgacgatttggctgaaaagcgaattgaaggcaatgatggaaaggacgtcacttggatcagtacagttattgaaggcgtcgaagaaatctcttggacgatctcccctgtcagtcttgatttatataatggcaatgcaggcatcggattttttatgagctatctgagccgcttcgcaaaacggccggagacttactcgcatataaccgagcagtgtgtatttgcgattcagcgagcgttgaatgaactgaaggaaaaagaagaattcctgaagtacgccgactctggggcattcacgggggtttccggctatctgtattttctgcagcatgcgggaacggttcagaaaaaaaacgaatggatcgaactcatacatgaagctctgccagtccttgaagctgtcatcgaacaagacgaaaactgcgatatcatcagcggttctgccggtgctctaatggttctgatgtcattgtatgaacaactggatgacccggtttttctaaagctcgccgaaaagtgcgccggccatttgcttcagcataaaacaaatattgaaaacggagcggcctggaaagatcctcatacacaaaactattacacaggatttgcccacggcacttccggcatcgccgcagctttatcccgattcaataaagtgtttgattcgcaatcactgaaaaaaatcatttcgcaatgcctggcatttgaaaagcagctgtacatcgcttccgaaaaaaattggggatcaaaaggaagagaacaactgtcagttgcatggtgccatggcgctgccggcatattgttgtcgagaagcatcctccgagaaaacggagtcaatgatcccggactgcataccgacatcttgaacgctcttgaaacaactgttaagcatgggctcggcaataaccgctcattctgtcacggcgatttcggccaactcgaaatcctaagagggttcagggaagaattcagcgaactgaacaccattatacagaatacggaagatcggctgttgacatattttcaagaaaatccattcagtaaaggggtatcacgaggtgtggattcagccgggctcatgcttggtttaagcggagtcggctacggcatgctgcaatgccaatatggagaagaactgccggaactgcttcagctcagtccgcctcaagcgcttatcaaaaagaacagcaaagcttttaaaagagaaaacgtgttttaa

SEQ ID NO: 42 - DNA, 2085 bp, artificial sequence
  PCR product “lig-PCR lan gene cluster flanks”
gtgctacgcgtacaacatgccaagaacagcacaacatgccaagaacagcagcagaggtgaagacatatggaacgaaaaaaagtcggcctgttagtcatggcatacggaaccccttatcaggaagaagatatcattccgtattacacgcatatccgtcatggaaaacgaccgtccgatgatatgattgaagacttgaaaaaacgctacaagcatatcggcggaatctctccgctcgcgaaaattacgctcgctcaggcaaaagagctggaaaaaacgttgaacgagcgccaagataaagtcgaatacgtgatgtatctcggcttaaaacacatctctccgtttatcgaggatgcggttgaacaaatgaaaaaagaccaaattgaagaggccgtttccgtcgtcttagctccccactattccaccttcagcaccgaagtatacaatcggcgcgccaaacaggccgcagcagcaatcggcggaccacggatcgcatcgattgacgagtggtatcaggaagaaggctttattcgctattggtctgaagaaatcggcagcattttaaacgacatgtctgaaaaagagcgggaaaaagcggccgtcatattctcagcacacagcctgccggaaaaaatcagggagcatgacgatccttacccggatcagctcgagaaaacagcacaactaatcggagaacggctgtcatttgaccagatcgccgtaggctggcaaagcgagggcaacacgcccgatccttggctcggcccggacgtccaagacttaacaaaggaactgtatgaagaaggcttccggtcattcatttacgcaccagtcggttttgtatctgaccacctggaagttctgtatgacaacgactatgagtgcaaagtggttacggacgagcttggcgcaagctatcaccgtccgcctatgccgaacactgatccgcggttcatcgatacgcttgcttcagtcgtggagcgaacatacaacagcacagaacaggagaaagcggagctgtaaatcgagctccgctttttgctgcaatggatccaatcaaaatctatggattttcatccatagattttttttgcaacattacgatgaaagaccttcatccaaatgcgcttattctcagattcgtgtcatttggcataatcaccagcttatgagcaaatccaactcacaacatgattattcagacaattcagaaatatatgctatgctctctctgattccaataaaagggagggatgatcggtgcccaccattcagtatccgccgtaattgtaagcgcttcccattcagcgttggcatcaaaaaagaaacaataggaggaatataatgaagaaattaatcagcatcatctttatctttgtattaggggttgtcgggtcattgacagcggcggtttcggcagaagcagcttctgccttaaactcgggcaaagtaaatccgcttgccgacttcagcttaaaaggctttgccgcactaaacggcggaacaacgggcggagaaggcggtcagacggtaaccgtaacaacgggagatcagctgattgcggcattaaaaaataagaatgcaaatacgcctttaaaaatttatgtcaacggcaccattacaacatcaaatacatccgcatcaaagattgacgtcaaagacgtgtcaaacgtatcgattgtcggatcagggaccaaaggggaactcaaagggatcggcatcaaaatatggcgggccaacaacatcatcatccgcaacttgaaaattcacgaggtcgcctcaggcgataaagacgcgatcggcattgaaggcccttctaaaaacatttgggttgatcataatgagctttaccacagcctgaacgttgacaaagattactatgacggattatttgacgtcaaaagagatgcggaatatattacattctcttggaactatgtgcacgatggatggaaatcaatgctgatgggttcatcggacagcgataattacaacaggacgattacattccatcataactggtttgagaatctgaattcgcgtgtgccgtcattccgtttcggagaaggccatatttacgcgttgtgc